(19) United States
(12) Patent Application Publication
(10) Pub. No.: US 2016/0008130 A1
(43) Pub. Date: Jan. 14, 2016

(54) ADJUSTABLE ANNULOPLASTY APPARATUS

(71) Applicants: MOR RESEARCH APPLICATIONS LTD., Tel-Aviv (IL); RAMBAM HEALTH CORPORATION, Haifa (IL)

(72) Inventor: Tal HASIN, Kochav Yair (IL)

(21) Appl. No.: 14/770,842

(22) PCT Filed: Feb. 27, 2014
(86) PCT No.: PCT/IL2014/050206

§ 371 Date: Aug. 27, 2015

Related U.S. Application Data
(60) Provisional application No. 61/770,480, filed on Feb. 28, 2013.

(51) Int. Cl. A61F 2/24

(52) U.S. Cl. CPC A61F 2/2445; A61F 2230/0069; A61F 2250/001

(57) ABSTRACT

An adjustable implantable annuloplasty apparatus suited for post-implantation adjustments according to changing needs of the patient. The apparatus comprises a ring shaped main body, and a designated adjustment section extending from the main body. The main body and designated adjustment section of a deployed apparatus are fully contained within a 5 cm diameter. Optionally, the apparatus is mechanically adjusted in a minimally invasive procedure, for example by engaging the designated adjustment section using a balloon catheter. Optionally, the apparatus is adjusted at least one month post implantation.

### RELATED APPLICATIONS

This application claims the benefit of priority under 35 USC §119(e) of U.S. Provisional Patent Application No. 61/770,480 filed Feb. 28, 2013 the contents of which are incorporated herein by reference in their entirety.

### FIELD AND BACKGROUND OF THE INVENTION

The present invention in some embodiments thereof, relates to an implantable device capable of being adjusted after it has been implanted in a patient. More specifically, some embodiments relate to an adjustable implantable annuloplasty apparatus which is suited for long term post-implantation adjustments according to changing needs of the patient.

The valves of the heart keep blood flowing in the proper direction through the heart. If the valves do not function properly, the blood circulation may be compromised. One of the methods to repair diseased or damaged atrioventricular valves is an annuloplasty procedure, in which a prosthetic repair segment or ring are attached to the annulus of the valve in order to support it and in some cases resize it, typically reducing its size.

When an annuloplasty ring is implanted in the human heart during open-heart surgery, its functioning can only be verified once the heart is resuscitated. A ring too small might cause post-operative valve stenosis, straining the heart and possibly reducing blood flow to the rest of the body. A ring too large might allow valve regurgitation (backward flow of blood from the ventricle into the atrium) to occur.

Following are disclosures of annuloplasty rings disclosed in previous publications.

US Patent Application Publication Number US2011/0230961 to Langer et al. discloses “A system for treating a cardiac valve includes an adjustable annuloplasty ring configured to attach to or near a cardiac valve annulus. The system also includes a suture comprising a first end coupled to the annuloplasty ring. A second end of the suture is configured to be anchored to a papillary muscle. Selectively adjusting the annuloplasty ring adjusts a tension of the suture to reposition the papillary muscle. The shape and/or size of the ring can dynamically adjust in response to a stimulus.”

US Patent Application Publication Number 2009/0216322 to Le et al. discloses “An intraoperative adjustment device useful for adjusting a size, dimension, or shape of an implanted annuloplasty ring substantially contemporaneously with the implantation of the annuloplasty ring or other adjustable device by applying energy to the annuloplasty ring appropriate for the adjustment thereof is disclosed. Also disclosed are methods for using the intraoperative adjustment device in the adjustment of an annuloplasty ring and an annuloplasty ring system. In certain embodiments, the annuloplasty ring is adjustable using an activation energy source, for example, radio frequency (RF) energy, microwave energy, ultrasonic energy, magnetic energy, electric energy, thermal energy, combinations thereof, and the like.”

U.S. Pat. No. 5,064,431 to Gilbertson et al. discloses “An annuloplasty ring of a tubular construction having two drawstrings, the ends of which extend from openings in the tube. The ring tube is stitched through the mid-section, defining two channels, the inner of which carries the drawstrings, and outer of which is used as a sewing flange for suturing the ring in place. A colored line along the top and bottom of the ring marks the stitching line, and, therefore, the dividing line between the channels. Each of the drawstrings is anchored at two anchor points, the anchor points alternating around the circumference of the ring to create a fixed nonadjustable zone between the center anchor points, two transitional adjustable zones, between the outside anchor point-pairs, each being controlled by one or the other of the drawstrings, and two adjustable zones between the outermost anchor points and the openings in the tube from which the drawstrings extend, each being controlled by both drawstrings, such that the drawstrings may be uniformly or selectively, symmetrically or asymmetrically tightened to adjust the annulus to a desired shape.”

### SUMMARY OF THE INVENTION

The present invention, in some embodiments thereof, relates to an implantable device capable of being adjusted after it has been implanted in a patient. More specifically, some embodiments relate to an adjustable implantable annuloplasty apparatus which is suited for long term post-implantation adjustments according to changing needs of the patient.

According to an aspect of some embodiments of the present invention there is provided an adjustable implantable annuloplasty apparatus including a ring-shaped main body aligning at least 50% of a human heart valve annulus, and a designated adjustment section for adjusting the main body, wherein said main body is shaped so that a lumen of said main body of a deployed apparatus coincides with a lumen of said heart valve annulus, and wherein at least a part of the adjustment section extends from the main body, and wherein the main body and the designated adjustment section of a deployed apparatus are fully contained within a 5 cm diameter. According to some embodiments of the invention, the main body and the designated adjustment section are mechanically adjustable in vivo. According to some embodiments of the invention, the designated adjustment section extends within the lumen of the main body. According to some embodiments of the invention, the designated adjustment section occupies only a portion of the lumen of the main body during adjustments to maintain an opening in which blood can flow through. According to some embodiments of the invention, the ring-shaped main body is non continuous and includes one or more gaps. According to some embodiments of the invention, the designated adjustment section is in the form of a loop. According to some embodiments of the invention, the loop is structured as a hollow tube. According to some embodiments of the invention, the loop includes an inner cord passing within the hollow tube. According to some embodiments of the invention, the apparatus includes at least one structural element which crosses the lumen of the main body. According to some embodiments of the invention, the structural element includes a rigid member. According to some embodiments of the invention, the rigid member includes at least one telescopic section, the telescopic section is extendable or compressible. According to some embodiments of the invention, the apparatus is collapsible to be delivered to the heart in a transapical catheterization procedure.

According to some embodiments of the invention, the adjustment section includes at least one radiopaque portion for visualizing the adjustment section using a fluoroscope. According to some embodiments of the invention, the designated adjustment section defines an aperture sized to surround a balloon. According to some embodiments of the invention, a lumen between the designated adjustment section and the main body is sized to receive a balloon. According to some embodiments of the invention, the main body includes a plurality of hooks for anchoring the main body to the tissue of the human heart valve annulus. According to some embodiments of the invention, at least one of the main body and the adjustment section include a restraining member for maintaining the shape of the main body. According to some embodiments of the invention, the restraining member includes a lock piece activated by application of mechanical force. According to some embodiments of the invention, the restraining member is mounted to an inner cord passing within a loop. According to some embodiments of the invention, the restraining member limits an adjustment of the main body to a predefined diameter.

In some embodiments, a position of the designated adjustment section with respect to a plane defined by at least a portion of the main body is controllable. In some embodiments, the main body comprises a ventricular facing side and an opposing atrial facing side, and the designated adjustment section extends away from the main body towards the volume of the atrium. In some embodiments, the adjustment section is formed, at least in part, of a soft flexible material which reduces damage to tissue the section may come in contact with. Optionally, a certain angle of the designated adjustment section with respect to the plane is selected from a range of 5-120 degrees. In some embodiments, the designated adjustment section is positioned away from the annulus so that it does not interfere with leaflets of a natural or prosthetic valve. In some embodiments, the designated adjustment section is rotatable on a hinge. Optionally, the hinge is a connector which couples between the designated adjustment section and the ring shaped main body. In some embodiments, the adjustment section is coupled to the main body by a tab coupled to an elastic element; wherein when the tab is received within a slot of the main body the elastic element is compressed to move the adjustment section away from the main body.

According to an aspect of some embodiments of the present invention there is provided a method for adjusting an adjustable annuloplasty apparatus including a ring-shaped main body and a designated adjustment section extending from the main body, including engaging the adjustment section inside the heart using a tool suitable for applying mechanical force, and adjusting the main body by applying mechanical force to reshape the heart valve annulus. According to some embodiments of the invention, the mechanical force is applied by inflating a balloon in contact with the designated adjustment section. According to some embodiments of the invention, the adjusting is performed during a catheterization procedure. According to some embodiments of the invention, the adjusting includes expanding the designated adjustment section for constricting the main body using a common inner cord passing within the adjustment section and the main body. According to some embodiments of the invention, the adjusting includes adjusting a multi-dimensional configuration of the apparatus.

According to some embodiment of the invention, the adjusting includes anchoring one or more hooks of the main body to the tissue of said human heart valve annulus. According to some embodiments of the invention, the adjusting includes unlocking a lock piece of a restraining member prior to adjusting. According to some embodiments of the invention, the adjusting includes incrementally advancing a lock piece on a rack mounted on an inner cord.

According to an aspect of some embodiments of the present invention there is provided a method for mechanically adjusting an implanted adjustable annuloplasty apparatus at least 1 month post implantation, including engaging an adjustment section of the apparatus inside the heart using a tool for applying mechanical force, and adjusting a main body of the apparatus by applying mechanical force to reshape the heart valve annulus at least 1 month post implantation.

According to an aspect of some embodiments of the invention there is provided a method for using an annuloplasty apparatus together with a prosthetic valve comprising leaflets and a supporting body, comprising implanting, in a heart of a patient in which an annuloplasty apparatus was previously implanted, a prosthetic valve; adjusting a main loop of said annuloplasty apparatus to match a contour of said supporting body of said prosthetic valve. In some embodiments, the method further comprises positioning a designated adjustment section of the annuloplasty apparatus away from the prosthetic valve. In some embodiments, adjusting comprises expanding or constricting the main loop of the annuloplasty apparatus. In some embodiments, the method further comprises expanding or constricting the supporting body of the prosthetic valve to match the main body of the annuloplasty apparatus. In some embodiments, constricting comprises reducing a size of one or more gaps between the main loop of the annuloplasty apparatus and the supporting body of the prosthetic valve to reduce blood leaks.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic diagram of an adjustable annuloplasty apparatus including a main loop and a designated adjustment loop, according to some embodiments of the invention;

FIGS. 2A-2E is a set of flow charts describing a general method of operation of an adjustable annuloplasty apparatus and a general method of use of an adjustable annuloplasty apparatus, according to some embodiments of the invention;

FIGS. 3A-3B is a set of flow charts describing a method for intentional adjustment of the main loop of the annuloplasty apparatus of FIG. 1, according to some embodiments of the invention;

FIGS. 4A-4B is a general view (FIG. 4A) and a cross section view (FIG. 4B) of an adjustable annuloplasty apparatus designed for a mitral valve annulus, according to some embodiments of the invention;

FIG. 5 is a schematic illustration of an annuloplasty apparatus being inserted into the human heart during implantation in a minimally invasive procedure, according to some embodiments of the invention;

FIG. 6 is a flow chart describing a post-implant adjustment method of an implanted annuloplasty apparatus using balloon inflation;

FIGS. 7A-7K is a set of illustrations showing a post-implant adjustment method of an apparatus implanted in the mitral valve annulus using balloon inflation, according to some embodiments of the invention;

FIGS. 8A-8D is a diagram showing various possibilities of shaping an apparatus designed for a mitral valve annulus, according to some embodiments of the invention;

FIGS. 9A-9D is a cross section view of relative changes in shape or diameter of an apparatus designed for a mitral valve annulus, according to some embodiments of the invention;

FIGS. 10A-10D is a general view of various configurations of an apparatus designed for the tricuspid valve annulus, according to some embodiments of the invention;

FIGS. 11A-11B is a detailed diagram of an annuloplasty apparatus designed for the tricuspid valve annulus, according to some embodiments of the invention;

FIGS. 12A-12B is a diagram showing various possibilities of shaping the apparatus of FIG. 11B, designed for the tricuspid valve annulus, according to some embodiments of the invention;

FIGS. 13A-13F is a side view of various configurations for an apparatus designed for the tricuspid valve annulus, and an illustration of a possible adjustment of the multi dimensional configuration of the apparatus, according to some embodiments of the invention;

FIGS. 14A-14I is a set of illustrations showing a post-implant adjustment method of an apparatus implanted in the tricuspid valve annulus using balloon inflation, according to some embodiments of the invention;

FIGS. 15A-15D is a diagram of various embodiments of adjustment units and elements, according to some embodiments of the invention;

FIGS. 16A-16E is a set of diagrams describing an apparatus comprising a telescopic pole, according to some embodiments of the invention;

FIGS. 17A-17C is a diagram of various embodiments of an apparatus comprising restraining members, according to some embodiments of the invention;

FIG. 18 is a diagram showing an apparatus including peripheral hooks for anchoring the apparatus to body tissue, according to some embodiments of the invention;

FIGS. 19A-19D show an annuloplasty apparatus comprising one or more adjustment sections that can be positioned with respect to the valve annulus and/or with respect to a main ring of the apparatus, and an exemplary movement mechanism of the adjustment section, according to some embodiments of the invention; and

FIGS. 20A-C illustrate a prosthetic valve and an annuloplasty apparatus implanted together in a heart, according to some embodiments of the invention; and

FIG. 21 is a flowchart of a method for using an annuloplasty apparatus together with a prosthetic mitral valve, according to some embodiments of the invention.

### DESCRIPTION OF SPECIFIC EMBODIMENTS OF THE INVENTION

The present invention in some embodiments relates to an implantable device capable of being adjusted after it has been implanted in a patient. More specifically, some embodiments relate to an adjustable implantable annuloplasty apparatus which is suited for long term post-implantation adjustments according to changing needs of the patient.

An aspect of some embodiments relates to an annuloplasty apparatus comprising a main body and a designated adjustment section. In an exemplary embodiment of the invention, the main body is ring-shaped and sized so that it can be used to align the valve annulus. Optionally, the adjustment section extends from the main body. In an exemplary embodiment of the invention, the adjustment section is positioned in proximity to the main body, such that the main body and the adjustment section of a deployed apparatus are fully contained within, for example, a 5 cm diameter.

In some embodiments of the invention, the adjustment section extends within a lumen of the main body. Optionally, the adjustment section occupies only a portion of the lumen of the main body of the apparatus during adjustments, so that the lumen is not blocked. Optionally, the adjustment section does not block the flow of blood through the valve annulus during adjustment of the apparatus, for example by the adjustment section and an adjustment mechanism (e.g. a balloon), together blocking less than 70%, 50%, 30%, 20% or intermediate percentages of said lumen and/or valve annulus.

In an exemplary embodiment of the invention, the apparatus comprises multiple adjustment sections. Optionally, two adjustment sections are provided, for example one for expanding the main body of the apparatus and the other for constricting the main body of the apparatus.

In some embodiments, an adjustment section, optionally additional, is defined by a lumen between the designated adjustment section (or other non-ring part of the apparatus) and the main body.

In some embodiments, the annuloplasty apparatus comprises a flexible (for example made of an easily bent material) loop. Optionally, the loop is attachable to human tissue. Optionally, the main body of the apparatus is a flexible loop. Additionally or alternatively, a designated adjustment section of the apparatus is a flexible loop. In some embodiments, an adjustment section is defined by a segment of a loop.

In some embodiments, the apparatus comprises a plurality of loops, for example one loop defining the main body and another loop defining an adjustment section. In some embodiments, components such as loops of the apparatus are independently adjustable. In some embodiments, components such as the loops of the apparatus are coupled together, for example by sharing an inner cord. Optionally, adjustment of a first loop or a loop segment results in the adjustment of a respective second loop or loop segment, for example by the use of a common element, for example by pulling on an inner cord that is passed in both loops or loop segments.

In some embodiments, components of the apparatus such as the main body are elastic, for example to withstand small deformations occurring naturally during the functioning of the heart. In some embodiments, the attachment of an adjustment section to the main body is rigid, for example by using a connector. Optionally, the connector reduces or prevents changes in the position of an adjustment section relative to the main body.

In some embodiments, a position of an adjustment section relative to the main body is controlled. Optionally, the adjustment section is moved with respect to the main body. Since, in some embodiments, the main body aligns the valve annulus, a position of the adjustment section relative to the valve annulus can be selected.

In some embodiments, the adjustment section is rotatable on a hinge, for example being a connector which couples between the main body of the apparatus and the adjustment section. Additionally or alternatively, the adjustment section is flexible, and can be moved with respect to its rotational axis. Optionally, a rotational axis of the adjustment section is a segment or a point in which the adjustment section is connected to the main body.

In some embodiments, a physician selects the position of the adjustment section with respect to the main body, for example during implanting and/or during adjusting of the apparatus. Optionally, the physician uses a catheter and/or a balloon to move the adjustment section into a certain position. Additionally or alternatively, the adjustment section is moved in response to adjusting a different portion of the apparatus, such as the main body.

In some embodiments, the adjustment section is positioned at a location in which it does not substantially interfere with inflating a balloon, for example inflating a balloon within the lumen of the main body. Optionally, the adjustment section is positioned such that it does not interfere with a prosthetic valve, for example with the leaflets of a valve, when a prosthetic valve and an annuloplasty apparatus are used together.

In some embodiments, the adjustment section is positioned at an angle with respect to a plane defined by at least a portion of the main body, such as a plane defined by an arc segment of the main body. In an example, the adjustment section is positioned on a plane perpendicular to the plane defined by the main body. Optionally, the adjustment section is movable from a position in which it lies within a plane defined by a main ring of the apparatus, to a position in which it is perpendicular to the annulus plane. In some embodiments, the main body is non planar.

In some embodiments, the adjustment section extends away from the main body, for example towards the volume of the atrium. Optionally, the adjustment section is positioned parallel to a direction of blood flowing through the annulus, so that it does, not interfere with the flow. Optionally, the adjustment section is formed of a flexible material which reduces the risk of causing damage to tissue such as an inner wall of the atrium.

An aspect of some embodiments relates to using annuloplasty apparatus together with a prosthetic mitral valve and/or prosthetic tricuspid valve. In some embodiments, an adjustment section of the annuloplasty apparatus is moved away from a plane on which the leaflets of the prosthetic valve are attached.

In some embodiments, the annuloplasty apparatus is adjusted, for example by expanding or contracting a main ring of the apparatus, to fit at least a portion of the prosthetic valve, such as a cylindrical stent-like portion of the valve. Additionally or alternatively, a stent-like portion of the prosthetic valve is expanded or contracted to fit the main body of the annuloplasty apparatus. Optionally, paravalvular leaks are reduced or prevented by matching the contours of the main body and the stent like portion of the valve.

An aspect of some embodiments relates to an annuloplasty apparatus adjusted by applying force on a part of the apparatus, for example applying force on an adjustment section. Optionally, the applied force is a mechanical force, such as radial force applied by the walls of an inflated balloon, which may be mounted on the tip of a maneuverable catheter.

In some embodiments, the apparatus or portions of it, such as the main body, are expanded as a result of applying a force. Additionally or alternatively, the apparatus or portions of it such as the main body are constricted as a result of applying a force.

In an exemplary embodiment of the invention, applying force includes engaging an adjustment section. Optionally, applying force includes directly contacting the adjustment section with, for example, a catheter and/or a balloon. Optionally, the mechanical force is applied from within a designated adjustment section. In some embodiments, the mechanical force is applied from within the main body.

In an exemplary embodiment of the invention, mechanical force applied by the walls of an inflated balloon inside a designated adjustment section may constrict the main body, for example by pulling on a common inner cord which passes through the main body and adjustment section, thereby increasing the portion of the cord passing within the adjustment section and decreasing the portion of the cord passing within the main body, resulting in constriction of the main body.

An exemplary method for adjusting an adjustable annuloplasty apparatus in vivo includes inserting a balloon catheter to a designated adjustment section of an implanted annuloplasty apparatus, and inflating the balloon.

In some embodiments, the balloon is inflated until a desired adjustment of the apparatus is obtained. For example expanding a loop of the main body to reach a predefined size to reshape the valve annulus. In some embodiments, the balloon is inflated a plurality of times. Optionally, the balloon is inflated in various sections of the apparatus. Optionally, a balloon is inflated and deflated multiple times, for example to advance a lock piece of a restraining member, for example as will be further explained.

In some embodiments, a standard (for example cylindrical) balloon is used for adjustments. Optionally, special shaped balloons (for example a cone shaped balloon or a balloon with a narrowing, such as a waist) are used. Optionally, the special shaped balloons are sized according to a designated adjustment section. Optionally, the shape of the special shaped balloons prevents the balloon from slipping out of an adjustment section during adjustments.

In some embodiments, the annuloplasty apparatus is implanted in the human heart using a minimally invasive surgical procedure, such as a percutaneous transapical procedure, or a transvascular procedure. In some embodiments, the main body of the apparatus is designed to fit a mitral valve annulus. In some embodiments, the main body of the apparatus is designed to fit a tricuspid valve annulus.

Optionally, the apparatus is collapsible into a compact form in order to be delivered by, for example, a catheter. Optionally, collapsing the apparatus to a compact form comprises folding the loops of the apparatus. Optionally, one or more loops are folded. Optionally, collapsing the apparatus to a compact form includes shortening a rigid member of the apparatus, such as a telescopic pole. Optionally, the main body and the adjustment section of the apparatus are reshaped when the apparatus is deployed at the valve annulus, for example the main body expanded.

An aspect of some embodiments relates to adjusting an annuloplasty apparatus post implantation, for example: immediately after the implantation, 3 weeks after the implantation, 6 months after the implantation, or 5 years after the implantation and/or at intermediate or later times. Optionally or alternatively, the apparatus is adjusted according to scheduled time periods. Optionally, the apparatus is adjusted according to the current needs of a patient, for example in cases in which a geometry of a patient's valve and/or heart changes. Optionally or alternatively, the apparatus is adjusted multiple times. Optionally, the adjustment is based on an estimated time period required for healing of the heart post implantation. Optionally, following the adjustment, the newly obtained configuration is maintained for an unlimited period of time, or until the apparatus is readjusted.

In some embodiments, different components of the annuloplasty apparatus, for example the designated adjustment section, are marked to be visualized under imaging and/or to be distinguished from the rest of the apparatus, for example the main body. Optionally, the markings enhance the visualization of the above components, for example during implantation or adjustment of the apparatus. Optionally, components are marked to indicate their location to a practitioner and/or to allow an imaging modality to detect them. Optionally, markings are located in areas that may assist determining a size of the marked component and/or to assist in guiding a balloon thereto, for example marking two ends of a diameter of an adjustment section. In an exemplary embodiment, components of the apparatus comprise a radio opaque material, such as platinum, to be visualized for example under fluoroscopy. In some embodiments, markings may be formed as dots, lines, or any other design that will facilitate detection. For example, a line may be used to mark a circumference of the adjustment section, and/or one or more dots may be placed at an extreme point of the adjustment section.

According to some embodiments, a multi dimensional structure of the apparatus is adjustable. Optionally, adjusting the structure comprises transforming an apparatus lying on a single plane to a three dimensional configuration. Optionally, adjusting the structure comprises extending or flattening the spatial layout of the apparatus. In an example, such adjustment can include bending out of plane (e.g., the general plane of the annulus). Optionally, the multi-dimensional structure is adjusted post implantation. Optionally, adjustment of the multi dimensional structure is obtained by reshaping only some portions of the apparatus, for example the main body, for creating a change in the multi-dimensional structure.

According to some embodiments, the annuloplasty apparatus comprises a structural element crossing the lumen of the main body of the apparatus. Optionally, the structural element is adjustable in length. Optionally, the structural crossing element is positioned along the coaptation line of the valve's leaflets. Optionally, the structural element is positioned at a non-zero angle to the coaptation line of the valve's leaflets, for example a 90° angle.

In some embodiments, the structural element comprises a rigid member, such as a rod. Optionally, the rigid member is used for stabilizing the apparatus, for example by restraining portions of the apparatus and/or preventing bending. Optionally, the rigid member is used for better fitting the apparatus to the destined anatomical site, for example the mitral or tricuspid valve annulus, such as by positioning the rigid member at the coaptation line. Optionally, the rigid member comprises a plurality of rods. Optionally, the rigid member divides the apparatus, for example the main body, into portions. Optionally, these portions are used as adjustment sections.

In some embodiments, the rigid member is a telescopic pole. Optionally, the telescopic pole is shortened, for example during implantation. Optionally, the telescopic pole is extended, for example during positioning of the main body at the valve annulus. Optionally, the length of the telescopic pole is adjusted in a post-implantation procedure. Optionally, the length of the pole is adjusted by applying mechanical force in a like manner to the method used for adjusting other parts of the apparatus, for example by inflating a balloon in a loop or loop segment connected to the ends of a pole, to lengthen the pole.

In some embodiments, the crossing structural element has a non-rigid form, for example being formed as a string or cable. Optionally, the cable limits adjustment of the apparatus, for example by restricting expansion of a loop. Optionally, the cable divides the apparatus into portions.

According to some embodiments, a loop's circumference is formed as a hollow tube, for example to allow an inner cord to be passed through the tube. Optionally, a single inner cord passes within more than one loop or loop segment to couple components together. In some embodiments, the common inner cord maintains its length during manipulation, enabling adjustment of two or more coupled loops by adjusting for example one of them, with the cord thereby forcing the adjustment of the other.

Optionally, a locking or restraining member maintains the cord in the adjusted configuration.

In some embodiments, the inner cord is used for setting the configuration of the apparatus (such as the multi-dimensional structure) prior to implantation. Optionally, the apparatus is shaped by deforming an inner cord, for example made of titanium. Optionally, the inner cord is adjusted post implantation for changing the configuration of the apparatus in vivo. Optionally, the inner cord is locked in position, for example by a restraining member such as a connector, for example to prevent unwanted changes such as expansion of the main loop. Optionally, the inner cord passes within the main body and within the adjustment section. Optionally, multiple inner cords are used.

According to some embodiments, connectors are used for adjoining the main body and adjustment section of the apparatus. Optionally, connectors are used for adjoining the flexible loops and/or the rigid member. Optionally, a connector adjoins two or more loops or loop segments to each other. Additionally or alternatively, a connector adjoins one or more loops or loop segments and the rigid member. Optionally, a connector is shaped to allow loops (and in some embodiments inner cord) to slide through the connector when adjustments are taking place. Optionally, when no force is applied, the connectors assist in maintaining the current shape and/or size of the loops, for example by clasping an inner cord to prevent it from sliding through.

In some embodiments, segments of the loops are connected by a pivot point, such as a pivot pin. Optionally, the pivot point adjoins the loops or segments of the loops in a movable manner. Optionally, the pivot point allows multiple loops or loop segments to be passed therethrough.

According to some embodiments, the apparatus includes a restraining member. Optionally, the restraining member maintains a current configuration of the apparatus. Alternatively, the restraining member enables only certain adjustments to take place. Optionally, previously described elements such as the structural crossing element, the connector or the inner cord function as restraining members, for example a connector which clasps the inner cord to prevent its movement. Optionally, the restraining member comprises a lock piece, which is released, (or transferred to an open position), for example prior to adjustments.

In an exemplary embodiment of the invention, the restraining member is activated by applying mechanical force, for example by using a balloon catheter. Optionally, activating comprises transforming the restraining member to an open or locked position.

In an exemplary embodiment, a restraining member includes ratchet like elements such as a toothed rack and pawl. Optionally, the ratchet like mechanism allows only certain adjustments to take place. Optionally, the adjustment is in a specific direction only, for example expansion. In some embodiments, the toothed rack is mounted to the loops and/or segments of the loops and/or to an inner cord passing through the tube of the main body and/or to the adjustment section. Optionally, the pawl is incrementally advanced on the toothed rack by inflating a balloon multiple times to push the pawl.

According to some embodiments of the invention, the main body of the apparatus includes anchoring elements, such as peripheral hooks, to fixate the main body of the apparatus to the tissue at the valve annulus. Optionally, the hooks are pushed into the tissue when force is applied to adjust the apparatus at the end of the implantation. Optionally, the hooks are uncoupled from the main body to allow adjustments to take place. Optionally, a tool used for inserting the apparatus during implantation (for example a catheter) is also used for uncoupling the hooks from the main body. Optionally, at the end of the adjustment, the hooks may be re-attached to the main body and/or to the tissue, for example using the delivery tool. Optionally, only a certain magnitude and/or direction of force are used to attach and/or uncouple the hooks.

In other embodiments, the apparatus includes one or more designated sewing locations for passing sutures.

In some embodiments, the apparatus comprises one or more rigid inner poles. Optionally, the poles are pushed away from each other and/or are brought closer to each other by applying force, for example by inflating a balloon.

In some embodiments, the apparatus is formed with one or more components that are rigidly attached to each other, for example enabling only certain deformations to take place.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

General Description and Use of an Adjustable Annuloplasty Apparatus According to Embodiments of this Invention

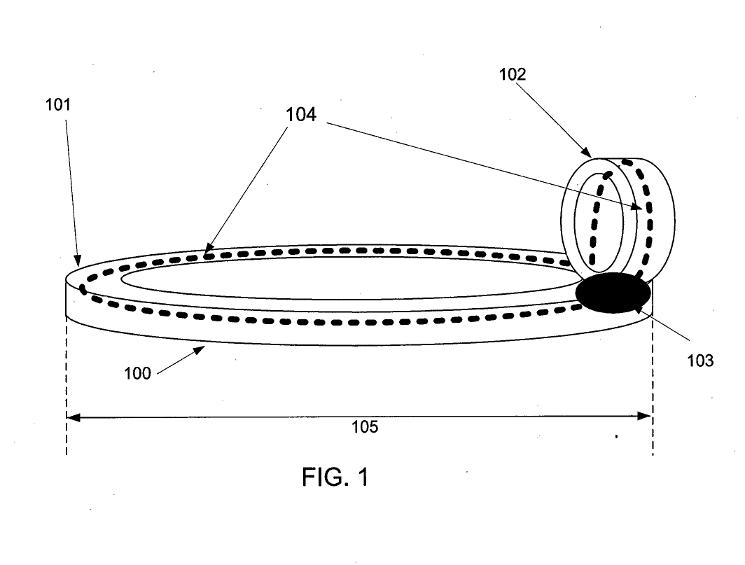

Reference is now made to FIG. 1, showing a schematic diagram of an adjustable annuloplasty apparatus 100 comprising a ring shaped main loop 101 defining the main body, and a designated adjustment section optionally formed as loop 102 extending from the main body, according to an embodiment of this invention. Optionally, in other embodiments, the main body is formed as a segment of a ring, for example suitable for being aligned with 50% or more of the valve annulus circumference, for example 75% of the valve annulus. Optionally, the main body is formed as a saddle, a D shaped ring, or any form that is in accordance to the anatomy of a patient's valve annulus. Optionally, the lumen of the main body of a deployed apparatus coincides with the lumen of the valve annulus.

Optionally, the designated adjustment section of the apparatus is, for example, in the form of a loop (as described in this figure), a segment of a loop, a saddle, overlapping free ends, or any other section which can be engaged by a tool for applying mechanical force.

Optionally, adjusting loops 101,102 includes adjusting a size, and/or shape and/or multi-dimensional configuration of the loops. Main loop 101 is adjustable, for example, by manipulation (such as expansion) of adjustment loop 102, for example if a common element such as an inner cord 104 passes within both loops, such that during adjustments the ratio of the portions of the inner cord passing within both loops changes (a portion of the cord lying within one loop is lengthened, and a portion of the cord lying within the coupled loop is shortened). For example, main loop 101 can be constricted by expanding adjustment loop 102. Constricting includes, for example, reducing a diameter (some embodiments may include 2 or more diameters), reducing a perimeter, or reducing a size of a portion of the apparatus. Expanding includes, for example, enlarging a diameter (some embodiments may include 2 or more diameters), enlarging a perimeter, or enlarging a size of a portion of the apparatus.

In some embodiments, adjusting includes changing the three dimensional configuration of the apparatus, for example by bending parts of the apparatus to be positioned in a different plane relative to other parts of the apparatus.

Optionally, the adjustment is performed by engaging the desired loop, for example adjustment loop 102, and applying force on the contacted loop. The applied force includes, for example, radial force created by the walls of an inflated balloon. Optionally, the force is applied from within the adjustment loop 102. Optionally, force is applied from within the main body 101. Optionally, once the loops are adjusted, apparatus 100 maintains the newly obtained shape or configuration for an unlimited period of time, or until reshaped again.

In the described embodiment, adjustment loop 102 is located on a perpendicular plane to the plane of main loop 101. In some embodiments, the designated adjustment section is positioned in the same plane as the plane defined by the main body (coplanar to the valve annulus). In some embodiments, the designated adjustment section is positioned in an intersecting plane to the plane defined by the main body, for example positioned in an intersecting plane with a 30° angle between the planes or an intersecting plane with a 60° angle between the planes. Optionally, adjustment loop is not constrained to a specific plane.

In some embodiments, the adjustment section is smaller than the main body, for example having a diameter that is one half, one third, or one quarter of a diameter of the main body. A diameter may be defined, for example, as the longest chord of a loop.

In some embodiments, the adjustment section extends within a segment of the lumen of the main body, for example occupying a region that is one half, one third, or one quarter of the total lumen area.

This figure shows a particular loop structure in which the loops are structured as hollow tubes, allowing the passing of an inner cord 104 through. Loops 101, 102 are connected through a connector 103 adjoining the loops in a movable manner, as to allow loops and/or inner cord 104 to be moved through the connector during adjustments. In some embodiments, inner cord 104 maintains its length for enabling adjustment of both loops 101,102 by adjusting only one of the loops.

Connector 103 can be, for example, a joint or tenon such as a screw, pin, band, strap, girth or any type of connector which can bind loops or segments thereof to other loops or loop segments, or, in some embodiments, to a rigid member. Optionally, when no force is applied, connector 103 assists in maintaining the current shape of the apparatus. Optionally, the connector includes an inner lock component.

Optionally, loop 101 and/or loop 102 is constructed of a flexible or a semi-flexible material, such as polyacetal, polyethylene, acetal resin or other elastic polymer. Optionally, the semi flexible material allows the apparatus to withstand small deformations occurring naturally during the functioning of the heart. Optionally, the loops are covered with a needle penetrable material, such as woven polyester, to facilitate suturing of the apparatus to the body tissue at the time of implantation. Inner cord 104 may be made of stainless steel, nitinol or Eligloy alloy or a different biocompatible metal or rigid material. Optionally, the inner cord is a braided cable.

In some embodiments, the apparatus or portions of it such as the main body are deformable. Optionally, for example in this figure, deforming the apparatus is done by changing the configuration of the inner cord, for example bending a portion of it. Optionally, the inner cord is strong enough as to not be affected by natural forces in the heart (for example forces created by the flow of blood) and to maintain the current configuration of the apparatus.

Optionally, the size of the outer dimensions of the main body of the apparatus is in accordance with the typical anatomical size of the valve annulus. Optionally, the apparatus is manufactured in different sizing. Optionally, the sizing is determined according to the anatomy of the valve annulus of a specific patient. Typical initial sizing of the long-axis 105 of the main loop 101 is, for example, 20 mm to 40 mm for mitral valves.

The size of the apparatus is measured, for example, as the diameter of the main loop 105, or as a total diameter of a plurality of loops in the annuloplasty apparatus.

In some embodiments, main loop 101 on its own is implanted to reshape the valve annulus.

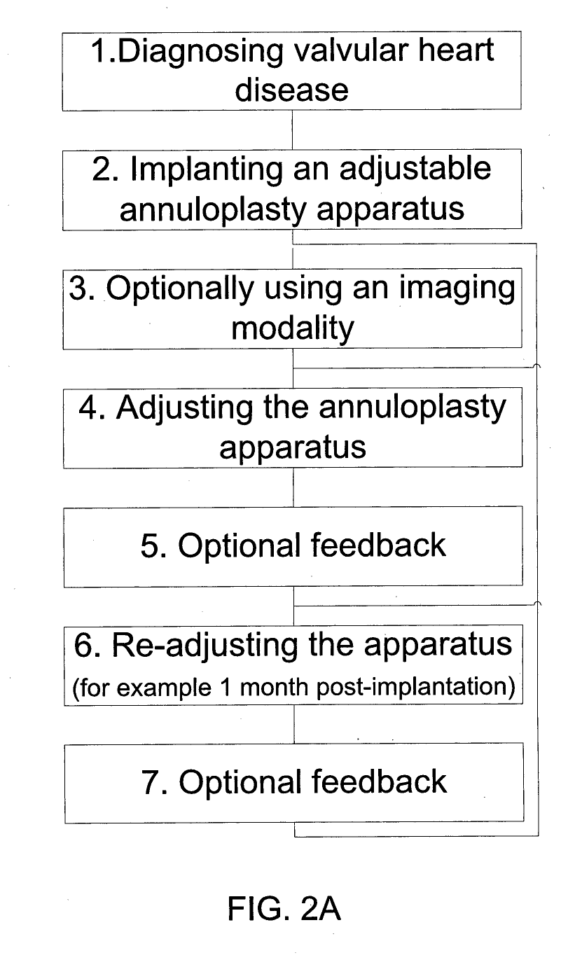

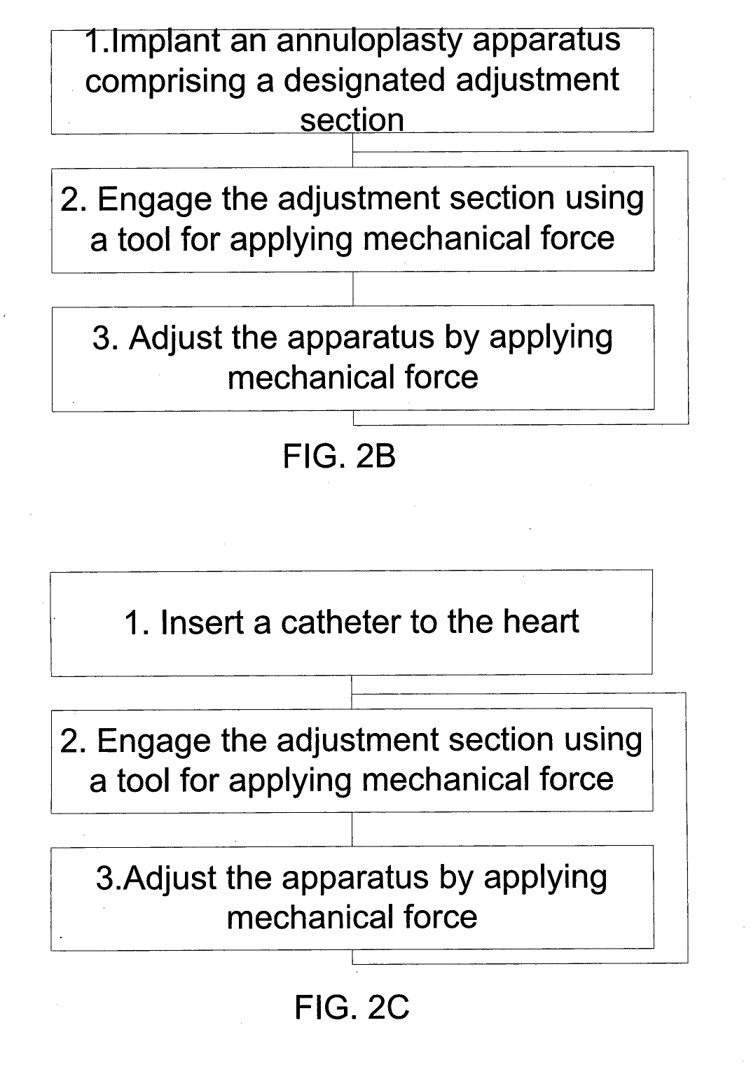

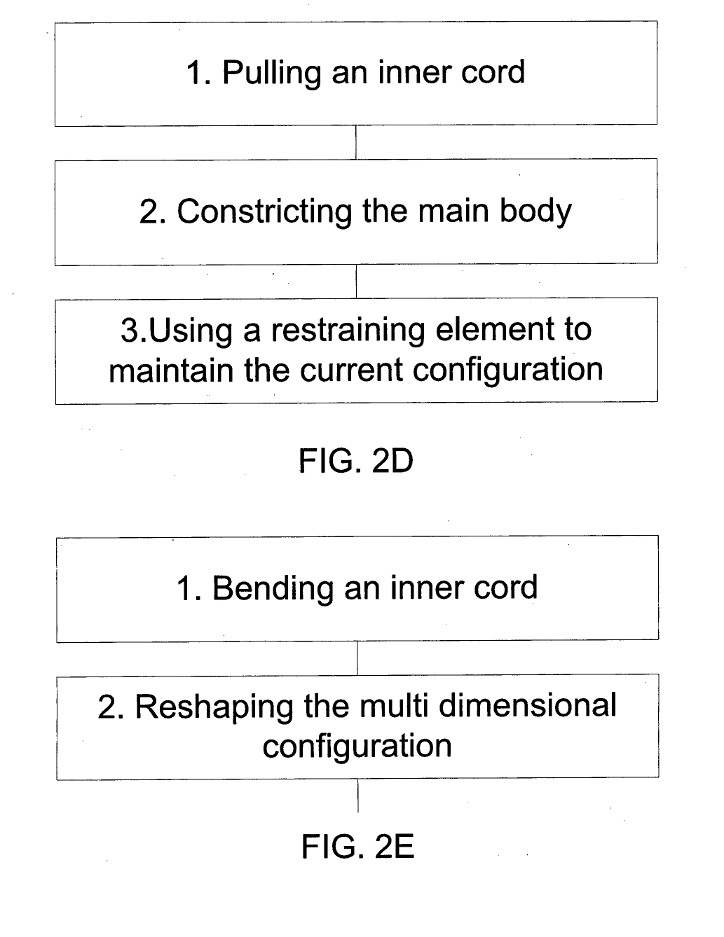

A general method of operation of an annuloplasty apparatus and a general method of use of an annuloplasty apparatus in accordance with an exemplary embodiment of the invention are shown in the flowcharts of FIG. 2.

In FIG. 2A, at block 1, a patient is diagnosed with valvular heart disease, for example mitral valve regurgitation, characterized by a backward flow of blood from the ventricle into the atrium. An adjustable annuloplasty apparatus may be implanted at the valve annulus, described at block 2, for example during a minimally invasive procedure (such as a transapical procedure), in order to reshape the annulus to reduce or prevent regurgitation and to allow normal blood flow through the valve.

Optionally, as described at block 3, an imaging modality such as fluoroscopy and/or echocardiography is used to facilitate guiding the apparatus to the mitral valve annulus and/or to determine the optimal configuration of the apparatus. Once the apparatus is implanted, it is possible to adjust its shape, size and/or multi-dimensional configuration, for example to expand the main loop of the apparatus to enlarge the passage through the annulus.

Adjusting the apparatus, as described at block 4, is optionally performed during a percutaneous procedure, such as catheterization. A tool for applying a mechanical force, for example an inflated balloon mounted at the end of a catheter, may be used to apply force on a designated adjustment section by engaging it, for example, from within the lumen. Optionally, adjustments are performed until an optimal configuration of the apparatus is reached and the functioning of the valve is repaired, possibly restoring normal blood flow through the valve. Optionally, normal (or within normal or desired parameters) functioning of the valve is assessed by using imaging modalities such as echocardiography, angiography, and/or hemodynamic catheterization, for providing feedback after a newly acquired adjustment, as described at block 5.

Additional adjustments, for example 1, 5, 10 adjustments, may be performed over time, for example 3 weeks after the implantation procedure, 4 months, 2 years, and/or at intermediate or later times, as described at block 6. Optionally, the apparatus is adjusted based on an estimated time period for healing of the heart post implantation. Optionally, the apparatus is adjusted if the geometry of the patient's valve changes. Optionally, the apparatus is adjusted if regurgitation or stenosis reoccurs. Optionally, feedback is provided once a new adjustment is performed, for example by using imaging modalities such as echocardiography, angiography and/or hemodynamic catheterization, as described at block 7. Optionally, the method as described at blocks 3-7 or any part of it is repeated, optionally a plurality of times, optionally to reach an optimal configuration (for example one that prevents regurgitation) of the implanted annuloplasty apparatus.

The flowcharts of FIG. 2B and FIG. 2C include a general method of use of the annuloplasty apparatus. FIG. 2B includes an initial method of use. FIG. 2C includes a secondary method of adjusting an implanted apparatus.

Block 1 of FIG. 2B describes implanting an annuloplasty apparatus comprising a designated adjustment section into the human body, for example at the mitral or tricuspid valve annulus.

The designated adjustment section of the apparatus is, for example, in the form of a loop, a segment of a loop, a saddle, overlapping free ends, or any other section which can be engaged by a tool for applying mechanical force, as described at block 2, for adjusting the apparatus.

In some embodiments, the main body of the apparatus is ring-shaped and sized so that it can be used to align the valve annulus, and the designated adjustment section extends from the main body, for example extends within the lumen of the main body.

In some embodiments, engaging the designated adjustment section includes contacting at least a part of the adjustment section directly using the tool. Optionally, engaging the designated adjustment section includes inserting the tool into a lumen of the adjustment section. Optionally, engaging the designated adjustment section includes fully occupying the lumen of a designated adjustment section. In some embodiments, an adjustment section, optionally additional, is defined by a lumen between the designated adjustment section (or other non-ring part of the apparatus) and the main body.

Optionally, a mechanical force is a force that results in movement of, for example, at least a part of a designated adjustment section, and/or at least a part of the main body of the apparatus, and/or in pulling or stretching at least a portion of an inner cord.

In some embodiments, a tool for applying mechanical force is a balloon, whereas the walls of an inflated balloon apply force on the adjustment section, for example radial force applied from within the adjustment section. In some embodiments, a tool for applying mechanical force is the tip of a catheter, which is used, for example, to push and bend an inner cord to change a configuration of the apparatus.

Once the tool engages the adjustment section, adjusting the apparatus, (as shown at block 3) is performed by applying force on the designated adjustment section. Optionally, adjusting the apparatus includes resizing it, for example constricting a main loop of the apparatus. Optionally, adjusting the apparatus includes reshaping a configuration, for example reshaping the apparatus from a flat single plane configuration to a three dimensional cone like configuration. Optionally, adjusting the apparatus includes adjusting only a portion of it, for example expanding only a portion of a main loop of the apparatus. Optionally, the process of engaging the designated adjustment section and applying mechanical force to adjust the apparatus is repeated a plurality of times.

As previously noted, adjusting the apparatus may be performed over time. A general method for adjusting the implanted annuloplasty apparatus, in accordance with an exemplary embodiment of the invention, is described FIG. 2C. Block 1 includes inserting a catheter into the heart to reach the implanted annuloplasty apparatus, for example at the mitral valve annulus. Optionally, the catheter is guided to the position of a designated adjustment section of the apparatus. Optionally, the catheter comprises a tool for applying mechanical force, for example an inflatable balloon. Block 2 and block 3 repeat the steps of engaging the designated adjustment section using the tool, and adjusting the apparatus by applying mechanical force. Optionally, the process of engaging the designated adjustment section and applying mechanical force to adjust the apparatus is repeated a plurality of times.

FIG. 2D describes a method of operation of the apparatus, in accordance with an exemplary embodiment of this invention. At block 1, an inner cord which passes within the tube of a loop of the apparatus is pulled and optionally shortened, for example to constrict the main body, as described at block 2, thereby reshaping the apparatus. For example, if the main loop of the apparatus is made of an elastic polymer such as silicon, the shortening of the inner cord may draw along the silicon body, thereby constricting the loop. Optionally, even if the main loop is sutured to the body tissue, the main loop is optionally squeezed through the sutures to allow constriction.

Optionally, at this point, the adjustment is reversible, and the main body may be expanded back to its original size, for example by lengthening the portion of the inner cord passing within the main body. In order to maintain the newly obtained adjustment, a restraining member, such as a lock piece is optionally used, as described at block 3.

Optionally, the inner cord is locked in its current position by the lock piece. Optionally, to resize the main body again at a later time, locking is released by, for example, inflating a balloon within an adjustment section, to apply force on the lock piece. Optionally, a balloon may be used for applying force on a lock piece, thereby overcoming the locking to enable further adjustments.

Optionally, adjusting the apparatus includes plastically deforming components of the apparatus. Optionally, adjusting the apparatus includes elastically deforming it.

Optionally, adjusting the apparatus includes both elastic and plastic deformations, for example subsequently.

FIG. 2E describes another method of operation of the apparatus, where the inner cord is used for setting a multi dimensional configuration of the apparatus. Block 1 describes bending an inner cord which passes within the tubes of the main body and/or within an adjustment section of the apparatus. Optionally, reshaping the apparatus is done by changing the configuration of the inner cord, for example bending a portion of it. Optionally, the inner cord is strong enough as to not be affected by natural forces in the heart (for example forces created by the flow of blood) and to maintain the current configuration of the apparatus, as described at block 2. Optionally, the inner cord is elastic or semi elastic, such as to enable slight deformations during the cardiac cycle and to return to its original desired configuration. Optionally, slight deformations of the inner cord may be allowed even when a restraining member is used for maintaining the current configuration.

Optionally, the device may be deformed at joints, for example connectors, for example between the main body and the adjustment section. Optionally, the device may be deformed along the tubes of the main body and/or adjustment section. Optionally, to maintain a configuration of the apparatus, components such as the inner cord or loops are locked in place, for example by a restraining member such as a connector that, for example, clasps them.

Optionally, a restraining member may prevent elastic recoil, for example of the main body, to a larger or smaller size. Optionally, a restraining member may prevent natural deformations of the heart from affecting the shape of the apparatus.

Optionally, as described above, operation of the apparatus includes plastically and/or elastically deforming at least parts of the apparatus. Optionally, a mode that combines both types of deformations includes advancing a pawl on a toothed rack which is mounted, for example, to an inner cord, as will be further explained.

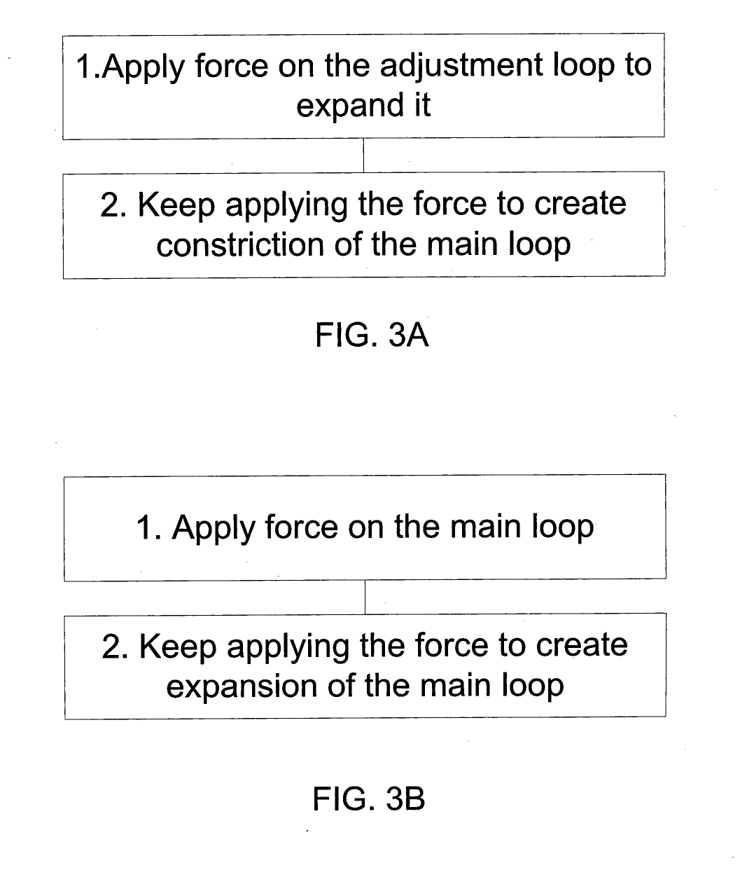

A method for intentionally adjusting an apparatus, in accordance with an exemplary embodiment of the invention, for example adjusting the main loop of the apparatus described in FIG. 1, is presented in FIG. 3. FIG. 3A is a flowchart describing intentional constriction of the main loop. At block 1 of FIG. 3A, force is applied (such as by the walls of an inflated balloon) on the adjustment loop to in order to expand it. If the inner cord maintains its length, the main loop will reduce in diameter. The force should be increasingly applied, as described at block 2, to reach a desired constriction of the main loop, for example to a predefined diameter.

In the same manner, the flowchart of FIG. 3B describes intentional expansion of the main loop. At block 1 of FIG. 3B force is applied (such as by the walls of an inflated balloon) on the main loop in order to expand it. If the inner cord maintains its length, the adjustment loop will optionally reduce in diameter. The force should be increasingly applied, as described at block 2, to reach a desired expansion of the main loop, for example to a predefined diameter.

### Embodiments of an Adjustable Annuloplasty Apparatus Designed for a Mitral Valve

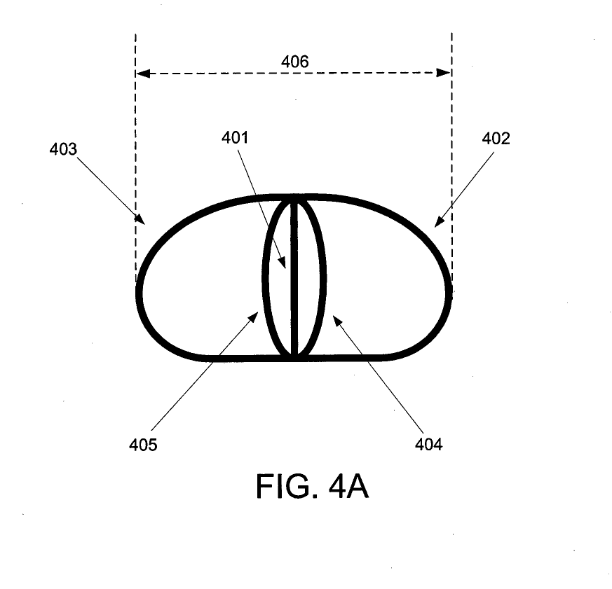

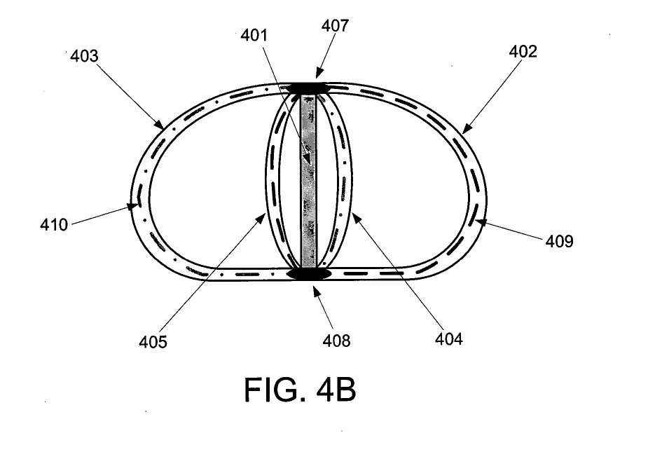

In accordance to an embodiment of this invention, the apparatus is designed to fit a mitral valve annulus, as shown in FIG. 4. The general view of the annuloplasty apparatus designed for the mitral valve is shown in FIG. 4A. The apparatus described in this figure includes a structural element which crosses the lumen of the main body. In this embodiment, the structural element is a rigid member—rod 401, which divides the apparatus into two main outer loops 402 and 403 defining the main body of the apparatus, and two inner adjustment sections 404 and 405. Optionally, in other embodiments, the crossing element is in a non-rigid form, such as a string or cable.

Optionally, the rigid member of a structural element includes multiple rods.

Optionally, the rigid member prevents portions of the apparatus from collapsing, for example preventing collapsing of opposite portions of the main body towards each other in a direction of the long axis, measured along dotted line 406.

Main outer loops 402 and 403 are complementary to form a D-shaped ring in accordance to the anatomy of the mitral annulus. The short axis of the D-shaped ring, measured along rod 401, are for example approximately 0.75 times the long axis of the ring, measured along dotted line 406.

A cross section view of the apparatus in a mitral valve design is shown in FIG. 4B. Main outer loops 402,403 and adjustment sections 404,405 are adjoined to rod 401 by connectors 407,408.

An inner cord passes inside the loops and their adjustment sections, such that inner cord 409 passes through main outer loop 402 and inner adjustment section 405. Similarly, inner cord 410 passes through main outer loop 403 and inner adjustment section 404. The coupling of the loops allows constriction or expansion of inner adjustment section 404 or 405 to result in constriction or expansion of the respective main outer loop 402 or 403, and vice versa.

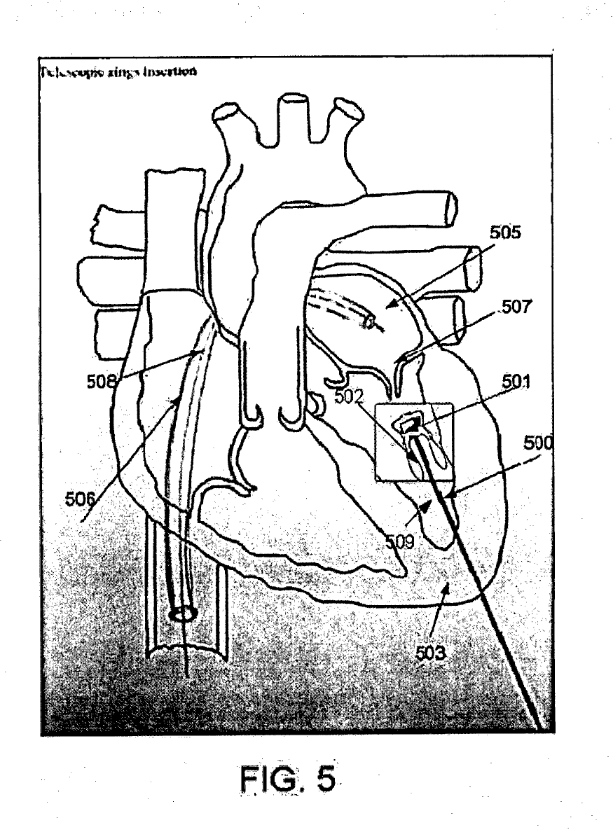

As previously noted, the apparatus can be implanted in the human heart during a minimally invasive procedure, for instance a transcatheter procedure. FIG. 5 shows a schematic view of inserting a collapsed apparatus delivered through a catheter towards the mitral valve annulus 507 during a minimally invasive procedure, for example a trasnsapical transcatheter procedure, in accordance with an exemplary embodiment of the invention.

Optionally, during insertion, the apparatus is collapsed to a compact form to be delivered through a catheter. Optionally, rod 501 of the apparatus (illustrated in this image in the embodiment of a telescopic pole, but may refer to any type of structural crossing element) is shortened. Loops 502 of the apparatus, including the main body and adjustment sections of the apparatus, are folded along the sides of rod 501. In some embodiments, loops are folded to surround the rod. In some embodiments, loops are folded multiple times to create an even more compact form of the apparatus.

The apparatus is guided by a catheter (or any type of insertion arm) 500 inserted from the direction of the apex of the heart 503 to reach the mitral valve annulus 507 from the left ventricle 509. Additionally or alternatively, the apparatus is manipulated by catheter 506 inserted through left atrium 505 to reach the mitral valve annulus 507 from the opposite direction. Optionally, a guiding wire 508 is inserted through catheter 506 and/or catheter 500. Optionally, both pathways (the transapical pathway and the atrium pathway) are used, for example one pathway for inserting the apparatus and the other for adjusting the apparatus in position. Optionally, catheter 500 and/or catheter 506 are shaped and size to allow delivery of the apparatus through. Optionally, the diameter of the catheter is sized according to a diameter of a collapsed or deployed apparatus. Optionally, the diameter of the catheter ranges between 9-14 F (3-4.7 mm). For example, the catheter diameter may be 9 F (3 mm) to deliver a collapsed apparatus having a diameter of, for example, one tenth of its original deployed diameter of the main body, which is, for example, 30 mm.

When the apparatus is guided to its deployed position at the mitral valve annulus 507, loops 502 and/or rod 501 are optionally unfolded and/or expanded to fit the annulus. Optionally, fitting adjustments are performed by applying force on designated adjustment sections of the apparatus, such as by the walls of an inflated balloon. Optionally, catheter (500, 506, or both) is used to push and direct parts of the apparatus to the desired position. In some embodiments, catheter (500,506 or both) is used to release restraining mechanisms, for example to unlock a lock piece to allow certain adjustments to take place, such as expansion. In some embodiments, catheter (500,506 or both) is used to couple and/or uncouple hooks intended to anchor the main body of the apparatus into the body tissue, in order to enable adjustment of the apparatus.

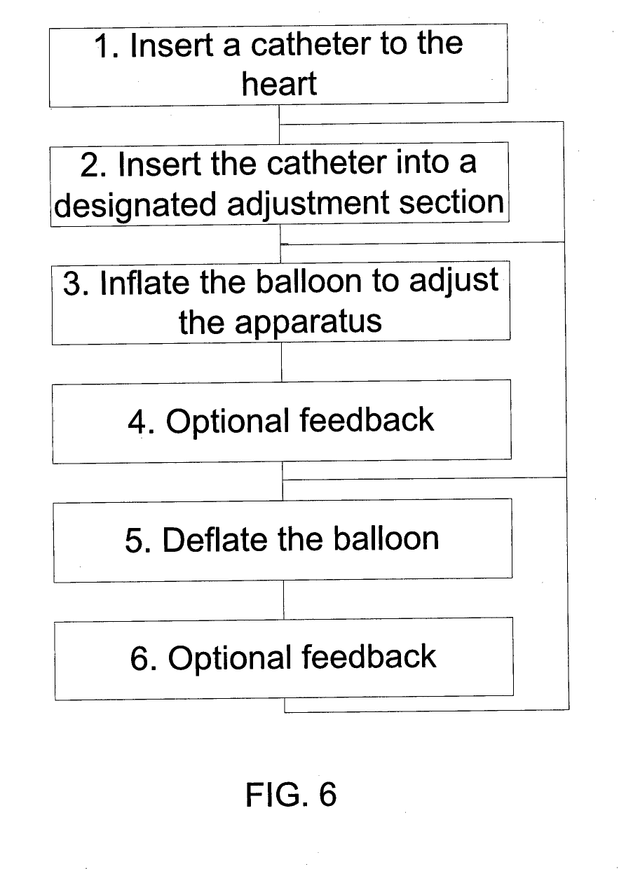

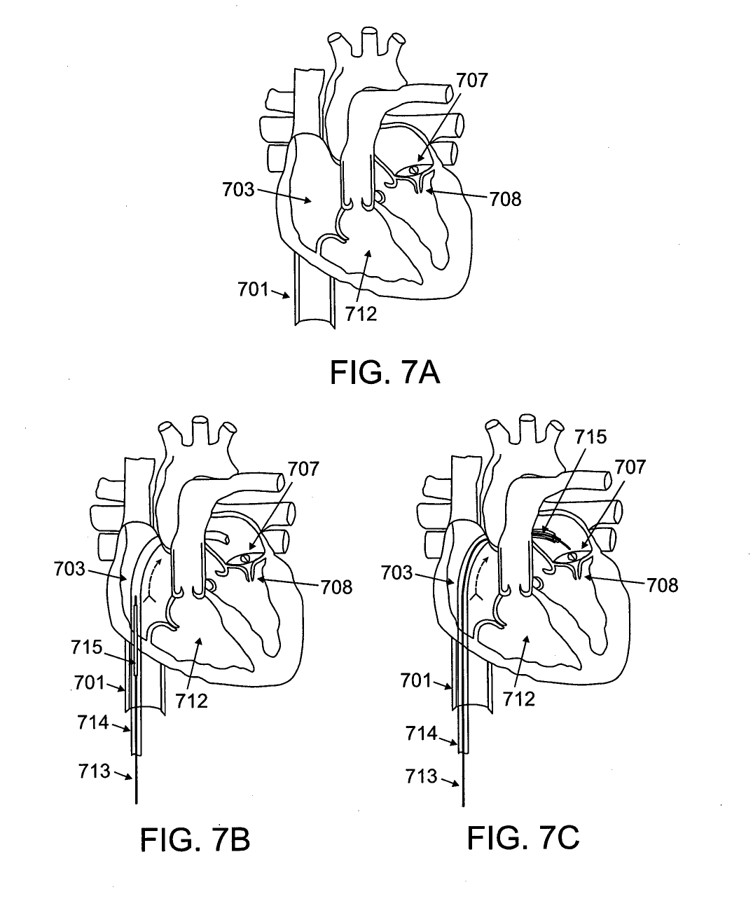

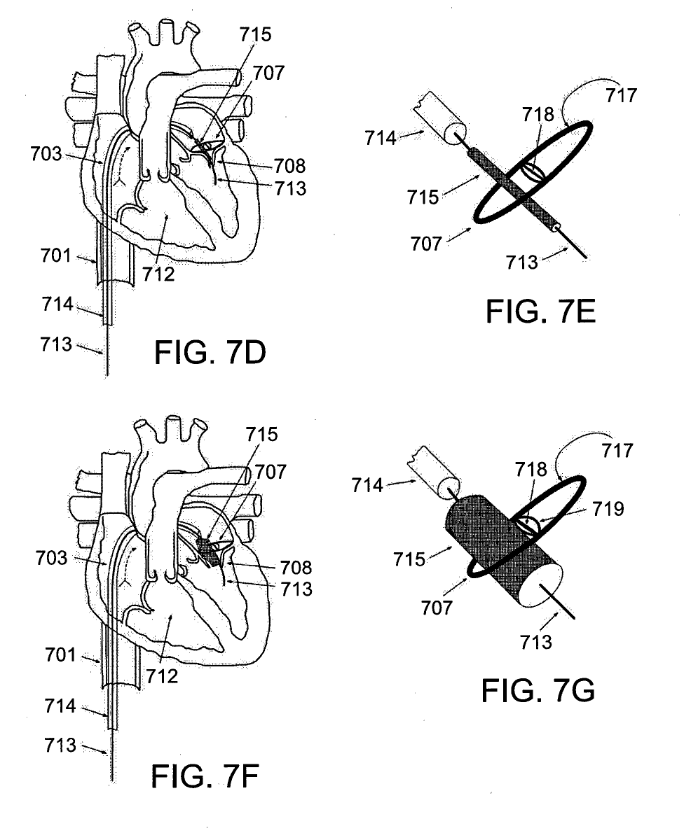

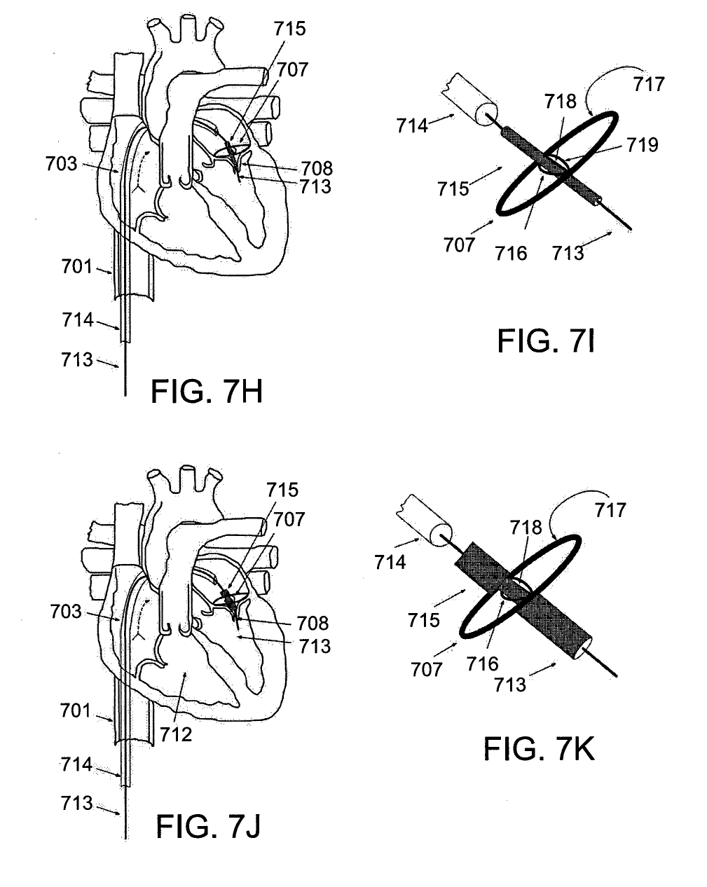

At the end of the implantation procedure, catheter (500, 506, or both) can be removed from the heart or used for further adjusting the apparatus, as described by the following FIGS. 6 and 7.

In the same manner, an embodiment of the apparatus designed for a tricuspid valve annulus may be implanted in the heart, guided by a catheter reaching through the direction of the apex of the heart and the right ventricle, and/or a catheter reaching through the right atrium in an opposite direction.

In some embodiments, the annuloplasty apparatus is implanted via an invasive procedure, e.g. open heart surgery. Optionally, in this type of procedure, the apparatus is inserted in either a collapsed or expanded form.

Optionally, the implanted apparatus is adjusted over time, if, for example, the geometry of the heart of a patient changes. Remodeling of the heart muscle geometry often occurs naturally after valve repair procedures, such as valvuloplasty, since the heart adjusts its function to the change in demand. Optionally, other procedures concomitantly performed, such as a bypass operation for stenosed coronary arteries or percutaneous coronary interventions may also cause for late effects on cardiac geometry and function, possibly raising the need for adjustments.

Once the apparatus is deployed at the valve annulus, it is possible to adjust its shape, size and/or multi-dimensional configuration, for example resize a diameter of the main body. Reference is now made to the flow chart of FIG. 6, and to FIG. 7, describing an exemplary post-implant adjustment method of the annuloplasty apparatus in a catheterization procedure using balloon inflation, in accordance with an exemplary embodiment of the invention. The method of FIG. 6 is described using the embodiment of the apparatus designed for a mitral valve, illustrated in FIG. 7.

In FIGS. 7A-7D,7F,7H,7J the anatomical structure of the human heart is illustrated, showing apparatus 707 implanted in the mitral valve annulus. FIGS. 7E,7G,7I and 7K illustrate detailed enlargements of FIGS. 7D,7F,7H,7J respectively. As described at block 1 of FIG. 6 and at FIG. 7, catheter 714 is inserted, optionally over a guiding wire 713 through the iliac vein, pushed through the inferior vena cava 701, the right atrium 703, and using septal puncture through the intra-atrial septum and into the left atrium to reach the implanted apparatus 707 at the mitral valve annulus. The two leaflets of the mitral valve are shown at 708. The right ventricle is shown at 712.

Balloon 715 is inserted through catheter 714 and over guiding wire 713. Guiding wire 713, followed by catheter 714 and balloon 715, is inserted (as also described at block 2 of FIG. 6) into one of the four available orifices, (optionally being adjustment sections), of implanted apparatus 707. Optionally, to facilitate detection of a specific orifice, parts of the apparatus such as an adjustment section are marked, for example with radiopaque material, to be easily identified by the practitioner when using an imaging modality such as fluoroscopy during adjustments.

FIGS. 7D-7E depict a side view of the insertion of a balloon 715 through an orifice between the left side of external loop 717 and rod 718 of apparatus 707.

FIG. 7F-7G depict the inflation of balloon 715 in the orifice between external loop 717 and rod 718 of the apparatus, as also described in block 3 of FIG. 6. Balloon 715 is inflated in the described section, and its inflated walls apply force on the left side of external loop 717. Optionally, the applied force causes expansion of the of the left side of external loop 717 (thereby expanding the outer circumference of the main body of the apparatus), and optionally reduction of the circumference of inner adjustment section 719, if the adjustment section 719 and left side of external loop 717 share a common element, such as an inner cord.

FIG. 7H-7I depict a side view of the insertion of a balloon 715 through the apparatus 707 into an orifice between inner adjustment section 716 and rod 718.

FIG. 7J-7K depict the inflation of a balloon 715 within the orifice between inner adjustment section 716 and rod 718. Balloon 715 is inflated in the described section, and its inflated walls apply force on the inner adjustment section 716. Optionally, the applied force causes expansion of inner adjustment section 716, and possibly constriction of to external loop 717. Optionally, in order to validate the functioning of the valve with the newly obtained configuration of the apparatus, feedback may be provided by using imaging modalities such as echocardiography, angiography and/or hemodynamic catheterization, for example to assess blood flow through the valve, as described at block 4. Optionally, to reach an optimal functioning of the valve, the adjustment procedure described at block 2-4 is repeated.

Once the apparatus is adjusted, the balloon is deflated as described at block 5 of FIG. 6, and optionally the catheter and wire are removed. Optionally, at this stage, feedback may be provided by using imaging modalities, for example to assess blood flow through the valve, as described at block 6. Optionally, the adjustment procedure described at blocks 1-6 or a part of it is repeated.

Optionally, adjustments are performed a plurality of times. Optionally, a balloon is re-inflated in the same orifice, for example to optimize the adjustment.

Depending on the flexibility of the inner materials and the outer materials forming the mitral apparatus 707, the outline shape of the main body of apparatus 707 may either be maintained in some embodiments, or changed in some embodiments, during the adjustment of the apparatus.

Optionally, balloons used for the adjustment of the apparatus are sized according to the designated portion of the apparatus in which they are inflated in. For example, a balloon used for adjusting the full diameter of a main body of an apparatus designed for example for a mitral valve, will inflate according to a diameter of 20-50 mm of the apparatus. A balloon used in a smaller adjustment section, for example, will inflate according to a 2-5 mm diameter of an adjustment section. A balloon used for example for expanding half of the main body of the apparatus will inflate according to a 10-25 mm diameter of an outer loop on one side of the apparatus.

In some embodiments, a standard (for example cylindrical) balloon is used for adjustments. Optionally, special shaped balloons (for example non cylindrical) are used. Optionally, the special shaped balloons are sized according to a designated adjustment section. Optionally, the shape of the special shaped balloons prevents the balloon from slipping out from an adjustment section.

In general, pressures generated by balloon inflation may reach for example up to 50 ATM. Pressures generated by balloon inflation within the heart valves may usually be in the range of 1-2 ATM only. Optionally, a pressure range of 1-5 ATM is required to force (by the walls of the inflated balloon) parts of the apparatus, to accomplish adjustments such as described above. Some embodiments allow maintaining the configuration of the annuloplasty apparatus when the balloon is inflated to a certain pressure, (e.g., pressure below a predetermined threshold), while inflating the balloon to a higher pressure may cause the walls of the balloon to apply a higher force on the apparatus, forcing it into a different shape, for example.

Echocardiography (trans-esophageal or trans-thoracic) or other imaging modalities are optionally used, for example simultaneously during the insertion of the catheter and adjusting the apparatus, to determine the optimal configuration of the apparatus. In some embodiments, different configurations of the apparatus are applied and imaged to determine an optimal configuration. Optionally, structural modalities such as MRI and/or CT are used before and/or during adjustments, to determine the optimal configuration of the apparatus.

In some embodiments, different components of the annuloplasty apparatus, for example the designated adjustment sections, are characterized with properties that distinguish them from the rest of the apparatus, for example the main body. Optionally, these properties enhance the visualization of the above components, for example during implantation or adjustment of the apparatus. Optionally, components are marked to indicate their location to a practitioner and/or to allow an imaging modality to detect them. For example, some components of the apparatus can comprise a radiopaque material, such as platinum, to be clearly visualized for example using fluoroscopy.

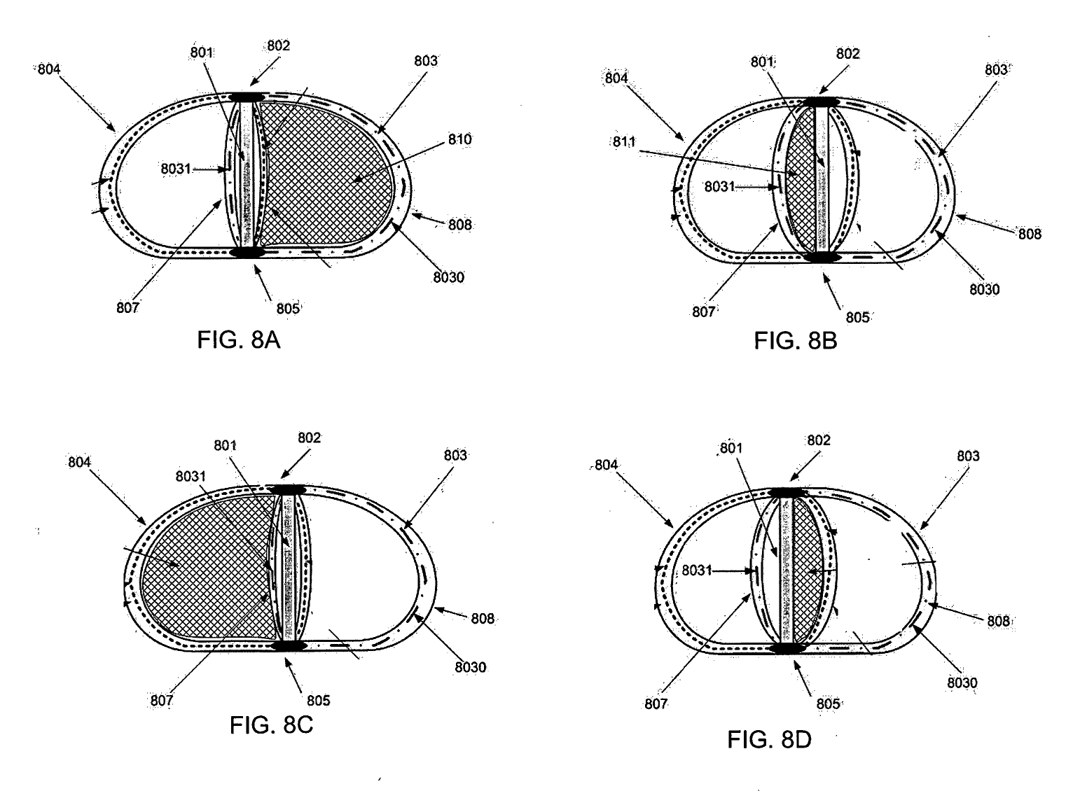

Some of the possibilities for shaping or re-shaping the implanted apparatus (for example the apparatus designed for a mitral valve described in FIG. 4), in accordance with an exemplary embodiment of the invention, are shown in FIG. 8. FIG. 8A depicts intentional expansion of the right side of the main body of the annuloplasty apparatus, comprising main outer loop 808. Inflation of balloon 810 between main outer loop 808 and rod 801 may lead to stretching or lengthening of inner cord 803, and the expansion of outer loop 808 laterally. In some embodiments, if inner cord 803 maintains its length (e.g., is not stretched), the ratio between outer loop segment 808 and inner adjustment section 807 changes as a result of applying force on the outer loop 808 as described.

A first portion 8030 of inner cord 803 lies within the tube of main outer loop 808, while a second portion 8031 of inner cord 803 lies within the tube of inner adjustment section 807. As main outer loop 808 is expanded due to the applied force, adjustment section 807 is therefore constricted (e.g., if main outer loop 808 is elongated by a centimeter, the result is that inner adjustment section 807 is simultaneously reduced in length by a centimeter).

In this exemplary configuration, two connectors 802 and 805 are positioned at both ends of rod 801. Optionally, connectors 802 and 805 couple between loops or loop segments, and/or between rod 801 and one or more loops.

FIG. 8B depicts intentional constriction of main outer loop 808 on the right side of the main body of the annuloplasty apparatus. Inflation of balloon 811 between inner adjustment section 807 and rod 801 may allow the stretching of inner cord 803 or elongating portion 8031 of it, thereby causing constriction of portion 8030 of cord 803 which lies within main outer loop 808, along with the expansion of inner adjustment section 807.

Similarly, FIG. 8C-8D illustrate expansion and constriction of main outer loop 804 on the left side of the main body annuloplasty apparatus.

Expansion and/or constriction of both main outer loop segments 804 and 808 may be combined simultaneously or subsequently, in order to adjust the annuloplasty apparatus according to the patient's mitral valve anatomy, and may cause expansion or constriction of the entire main body of the apparatus in some cases, or expansion of one side and constriction of another side of the main body of the apparatus in other cases.

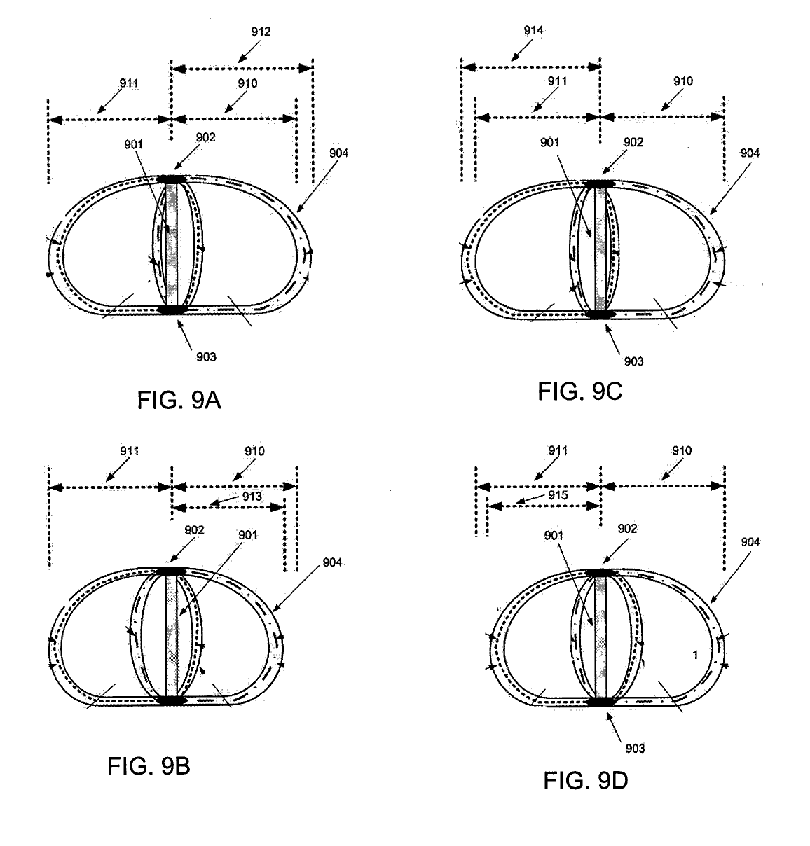

Optionally, the above described adjustments result in relative changes in, for example, the diameter of the main body of the apparatus, as shown in FIG. 9 FIG. 9A depicts relative expansion of right main outer loop 904 in accordance with an exemplary embodiment of the invention. New lateral margin of the main body extends beyond first diameter 910, which indicates an initial size of the main body. Diameter 912 indicates the new lengthened diameter of right main outer loop 904. Rod 901 is shown to extend between two connectors 902 and 903.

FIG. 9B depicts relative constriction of right main outer loop 904. The lateral margin 910 of the main body is reduced, such that adjusted diameter 913 is shorter than the previous (e.g. initial) diameter 910.

Optionally, in FIG. 9A, the initial diameter of the main body of the apparatus is measured by summing length 910 and length 911. The new diameter of the expanded main body of the apparatus is measured as the sum of length 911 plus length 912. Similarly, in FIG. 9B, the initial diameter of the main body of the apparatus is determined as sum of length 910 plus length 911, and the adjusted diameter is determined as sum of length 910 plus newly adjusted (e.g. shortened) length 913.

Similarly, FIG. 9C-9D illustrate the relative changes of expansion and constriction of the main body of the apparatus by adjusting the left side of the apparatus.

As previously noted, both sides of the main body of the apparatus can be adjusted (simultaneously or subsequently) to reshape and/or resize the apparatus.

### Embodiments of an Adjustable Annuloplasty Apparatus Designed for a Tricuspid Valve

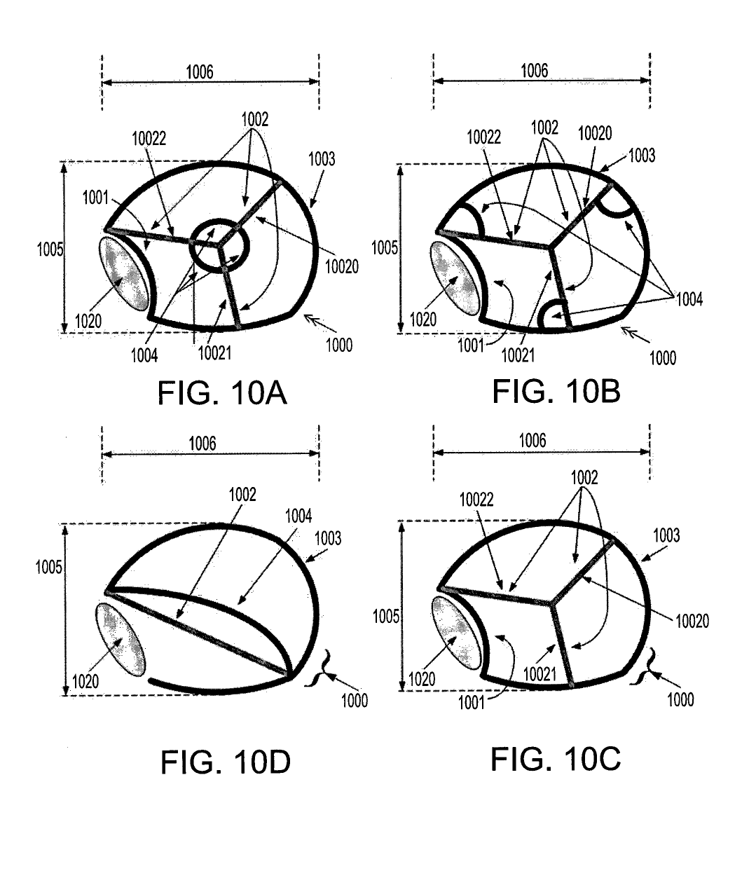

A general view of various configurations of an annuloplasty apparatus designed for a tricuspid valve annulus in accordance with an exemplary embodiment of the invention are shown in FIG. 10.

In the embodiments of FIG. 10A-10D, main body of annuloplasty apparatus 1000 is shaped in accordance to the anatomy of a tricuspid valve annulus. Optionally, the main body includes a protected, inwardly-indented recess or gap 1001 corresponding with sensitive area 1020 that includes the conductive system of the human heart located approximately in the medial part of the tricuspid valve. The outer margins of the main body of the apparatus, determined by main outer loops 1003, assist in forming a substantially D-shaped ring. The structure of the apparatus described in these embodiments also includes rigid member 1002, composed of one or more rod like elements.

As shown in the embodiments of FIG. 10A-10C, apparatus 1000 includes a rigid structural crossing element 1002 in the form of three elongated rod-like elements 10020, 10021, 10022, based substantially at the center of the apparatus 1000, and distributed evenly, for example with 120 degrees between the rod elements. In another embodiment of the invention, central rod like rigid element 1002 is in the form of a straight obscure diameter, as depicted in FIG. 10D. Adjoining the outer rods of central rigid member 1002 are a plurality of main outer loops 1003, defining the main body, which are optionally sutured to the annulus of the tricuspid valve. An inward indentation in the outer structure of the apparatus, 1001, is included and optionally avoids contact with sensitive zone 1020 approximate the medial part of the valve, as described above.

One or more designated adjustment sections 1004 can be fitted within the annuloplasty apparatus. In some embodiments, adjustment sections 1004 are fitted substantially in the central portion of the main body of the apparatus 1000 as depicted in FIGS. 10A and 10D. In some embodiment's, adjustment sections 1004 are fitted in the internal portion of the main body of the apparatus 1000, along the perimeter, as depicted for example in FIG. 10B. Some embodiments include a combination of central adjustment sections and peripheral adjustments sections.

Short axis 1005 of this substantially D-shaped ring, as depicted in FIG. 10A-10D, are slightly shorter than longer axis 1006, for example approximately 0.8 times the longer axis. Typical initial sizing of the longer axis 1006 of the D-shape is, for example, 20 mm to 50 mm for the tricuspid valve design. In some embodiments, the apparatus size is determined as a set of parameters, for example a first diameter and a second diameter. In one example, the first diameter may include the longer axis 1006, and the second diameter includes the shorter axis 1005. In certain embodiments, diameters or measurements of other segments of the apparatus may determine the size.

Optionally, adjustment sections 1004 are reshaped by applying force, for example by the inflation of a balloon. Optionally, reshaping an adjustment section results in adjusting the respective outer loop as well, if a common element such as an inner cord is used. Optionally, adjustments can be combined to create expansion of the outer loops 1003, or for example to constrict one loop and expand the other, or any other combination as such. In another embodiment, the circumference of the main body of annuloplasty apparatus 1000 can only be expanded, as depicted for example in FIG. 10C. Optionally, the apparatus will maintain the newly-obtained shape or the adjusted configuration for an unlimited period of time, or until it is reshaped.

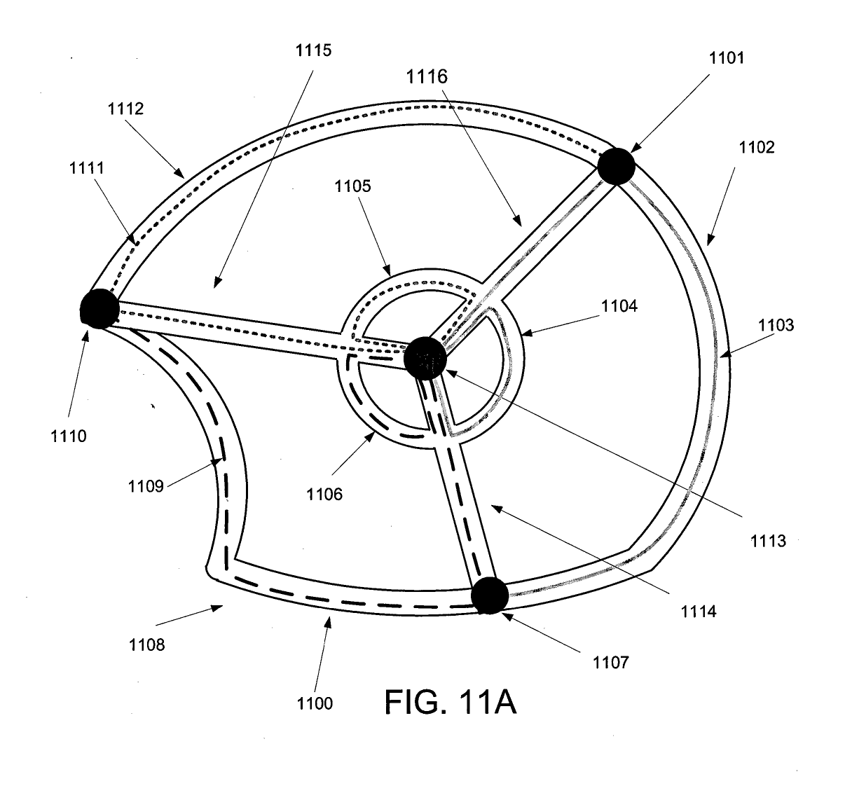

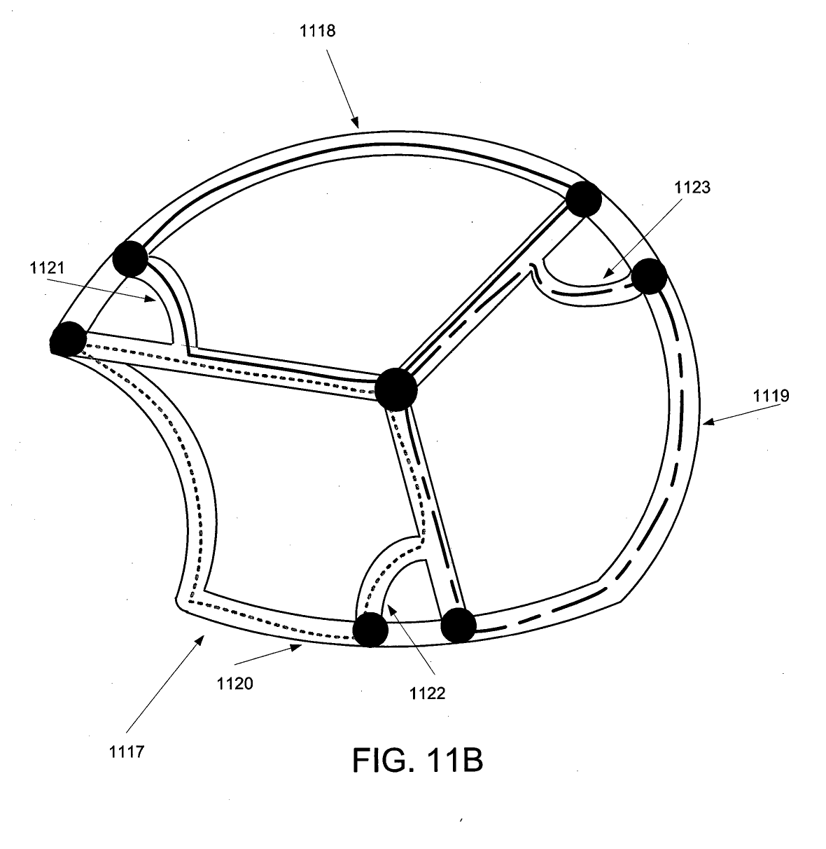

A more detailed structure of the apparatus in some of its embodiments designed for a tricuspid valve in accordance with an exemplary embodiment of the invention is presented in a cross section view in FIG. 11. FIG. 11A shows an apparatus including central adjustment sections 1104,1105,1106. Apparatus 1100 includes a rigid structural crossing element comprised of a plurality of rods, for example, three rods 1114, 1115, 1116, which are typically rigid or semi-rigid rods. Optionally, the rods maintain their structure, even if certain pressure is applied to them. Rods 1114, 1115, 1116 are joined together in the center or substantially in the center of the main body of the apparatus 1100 by connector 1113. Connectors 1101, 1107 and 1110 adjoin main outer loops 1102, 1108 and 1112, comprising the main body, to the rods.

Apparatus 1100 includes a main body, divided into a plurality of main outer loops, for example into three main outer loop loops 1102, 1108 and 1112. Each main outer loop 1102, 1108 and 1112 includes an inner adjustment section 1104, 1106 and 1105 respectively. For example, main outer loop 1102 is adjusted by manipulation of inner adjustment section 1104 that is located inside main outer loop 1102. Main outer loop 1108 is adjusted by manipulation of inner adjustment section 1106 that is located inside loop portion 1108, and main outer loop 1112 is adjusted by manipulation of inner adjustment section 1105 that is located inside it.

In the particular structure shown in this figure, main outer loops 1102, 1108 and 1112, the inner rods 1114, 1115,1116 and/or inner adjustment sections 1104, 1105, 1106 are structured as hollow tubes, allowing one or more inner cords 1103, 1109 and 1111 to pass through their hollow channels. A single inner cord may connect more than one component. For example, inner cord 1103 passes through outer loop 1102, rod 1116 and inner adjustment section 1104. Inner cord 1109 passes through outer loop 1108, rod 1114 and inner adjustment section 1106. Inner cord 1111 passes through outer loop 1112, rod 1115 and inner adjustment section 1105.

Connector 1113 adjoins the adjustment sections 1104, 1105, 1106 to the central rods 1114, 1115, 1116 in a non-rigid, movable manner, as to allow inner adjustment sections 1104, 1105, 1106 to be moved through the connectors, for example by applying certain force. Optionally, connectors 1113, 1101, 1107 and 1110 include inner locks, bolts or friction devices to stabilize inner cords 1103, 1109 and 1111. Optionally, when no force is applied, connectors 1113, 1101, 1107 and 1110 assist in maintaining the current shape, and/or size of main outer loops 1102, 108 and 1112 and inner adjustment sections 1104, 1105, 1106, thereby maintaining the ratio between the outer main loops and inner adjustment sections.

FIG. 11B shows an embodiment of the apparatus with peripheral adjustment sections. Apparatus 1117 includes, for example, three adjustable main outer loops 1118, 1119 and 1120 comprising the main body of the apparatus, each including an adjustment section 1121, 1123 and 1122 respectively, positioned on the perimeter of the external loops. For example, main outer loop 1118 is adjusted by manipulation of inner adjustment section 1121 that is located inside it. Main outer loop 1119 is adjusted by manipulation of inner adjustment section 1123 that is located inside it and main outer loop 1120 is adjusted by manipulation of inner adjustment section 1122 that is located inside it.

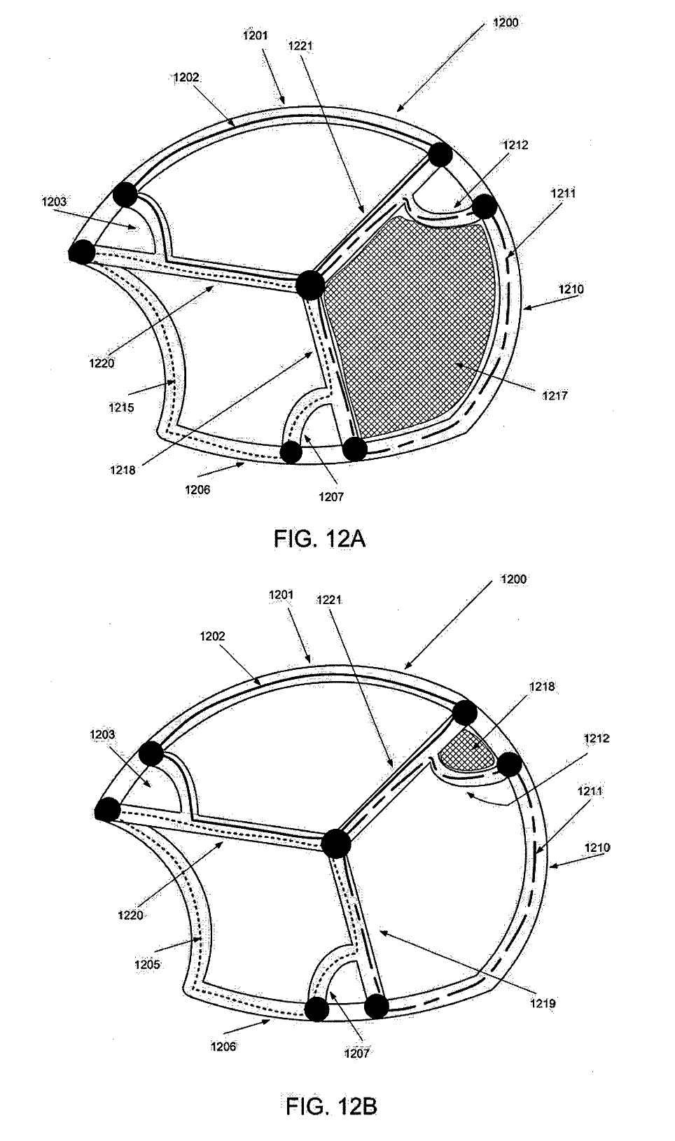

Various possibilities of shaping an apparatus designed for the tricuspid valve in accordance with an exemplary embodiment of the invention are illustrated in FIG. 12. FIG. 12A depicts intentional expansion of a portion of the main body of the annuloplasty apparatus. Optionally, inflation of balloon 1217 between main outer loop 1210 and rods 1219 and 1221, allows a change in the ratio with increased portion of cord 1211 within the outer loop 1210 and decreased portion in the adjustment section 1212. The balloon inflation may cause the lateral expansion of loop 1210. Optionally, inner adjustment section 1212 will reduce in size. Optionally, simultaneous or subsequent balloon inflation in two or more main outer loops 1201, 1206 and 1210 results, for example, in expansion of the main body of annuloplasty apparatus 1200.

FIG. 12B depicts intentional constriction of a portion of the main body of the annuloplasty apparatus 1200. Optionally, inflation of balloon 1218 between the inner adjustment section 1212 and rod 1221 allows a change in the ratio with increased portion of cord 1211 within adjustment section 1212, and decreased portion in main outer loop 1210, possibly creating constriction of loop 1210 with the expansion of inner adjustment section 1212. Optionally, balloon inflation in two or more inner adjustment sections 1212, 1203 and 1207 results, for example, in constriction of the main body of the annuloplasty apparatus 1200.

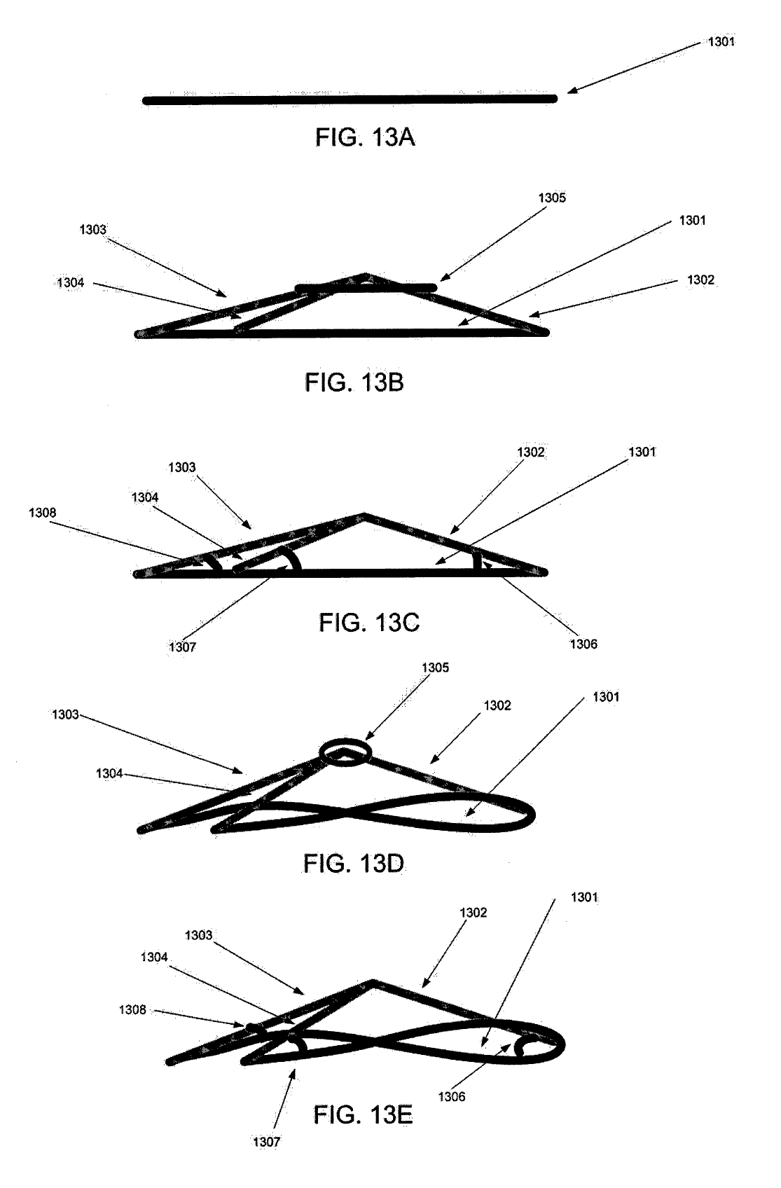

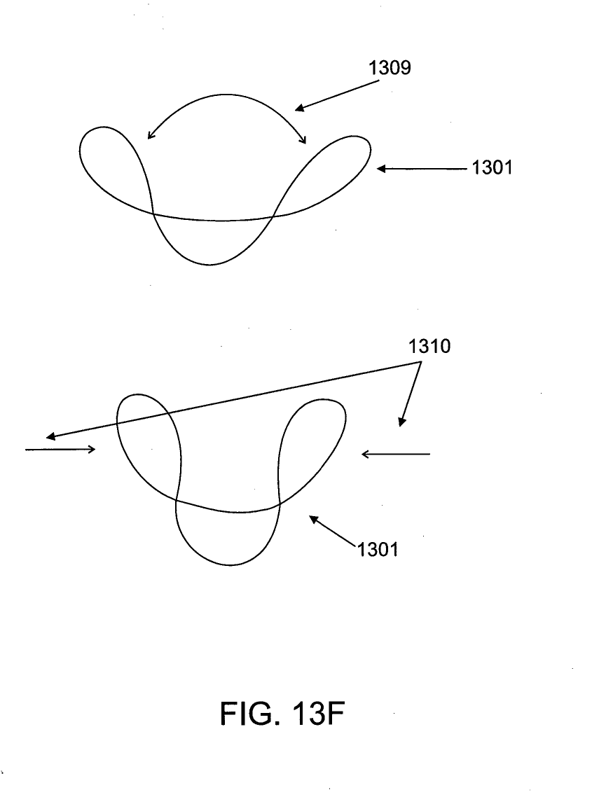

Various configurations of the apparatus designed for a tricuspid valve in accordance with an exemplary embodiment of the invention, are shown from a side view in the diagram of FIG. 13. Optionally, the configuration is adapted prior to implantation, for example by means of bending the inner cord. Optionally, the apparatus is flat and lies in a single plane, as seen in FIG. 13A. Alternatively, the apparatus is shaped in a three dimensional cone-like configuration, where the inner rods 1302-1304 are angled towards the main outer loops 1301, as seen in FIG. 13B and FIG. 13C. Such cone-shaped configuration may better facilitate the insertion of modification devices which are used for adjusting the apparatus, such as wires and balloons delivered over a catheter. Optionally, this cone like configuration is used with one or more central adjustment sections 1305, depicted in FIG. 13B, or with one or more peripheral adjustment sections 1306-1308, depicted in FIG. 13C, or with a combination of both central and peripheral in other embodiments.

FIG. 13D and FIG. 13E illustrate an embodiment in which the design of the main outer loops 1301 is formed in a curved fashion, which may better fit the structure of the tricuspid valve annulus. A similar embodiment could be combined with the central adjustment sections 1305 as depicted in FIG. 13D or with the peripheral adjustment sections 1306-1308 as depicted in FIG. 13E.

FIG. 13F illustrates a schematic view of an embodiment in which the multi-dimensional configuration of the apparatus is adjusted, optionally before or after the implantation, for example by applying force on the main outer loops 1301. Optionally, applying force in the directions shown by arrow 1309 results in flattening the loops 1301, for example to lie in a single plane. Alternatively, applying force in the opposite direction from 1309, shown by arrows 1310, results in bending the main outer loops upwards towards each other to receive a new configuration. Optionally, a catheter or any insertion tool is used to bend the apparatus to a new configuration. Optionally, an inflated balloon is used to bend the apparatus, when positioned to allow its inflated walls to apply force in the required direction. In some embodiments, as previously mentioned, the multi-dimensional configuration is adjusted by bending an inner cord.

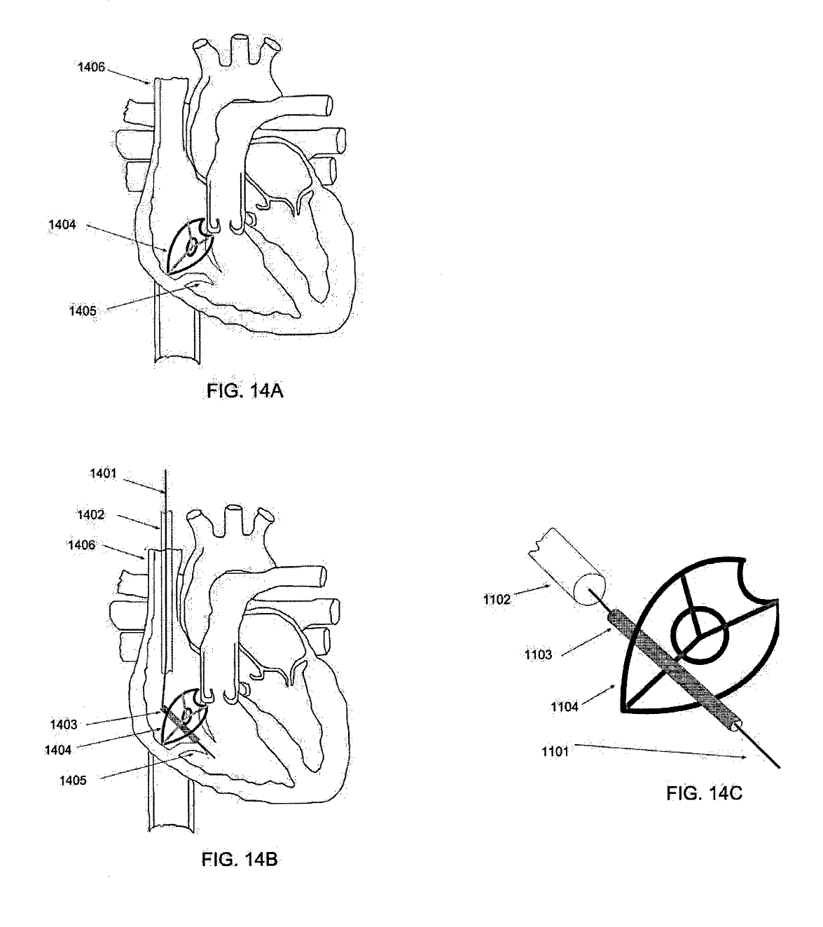

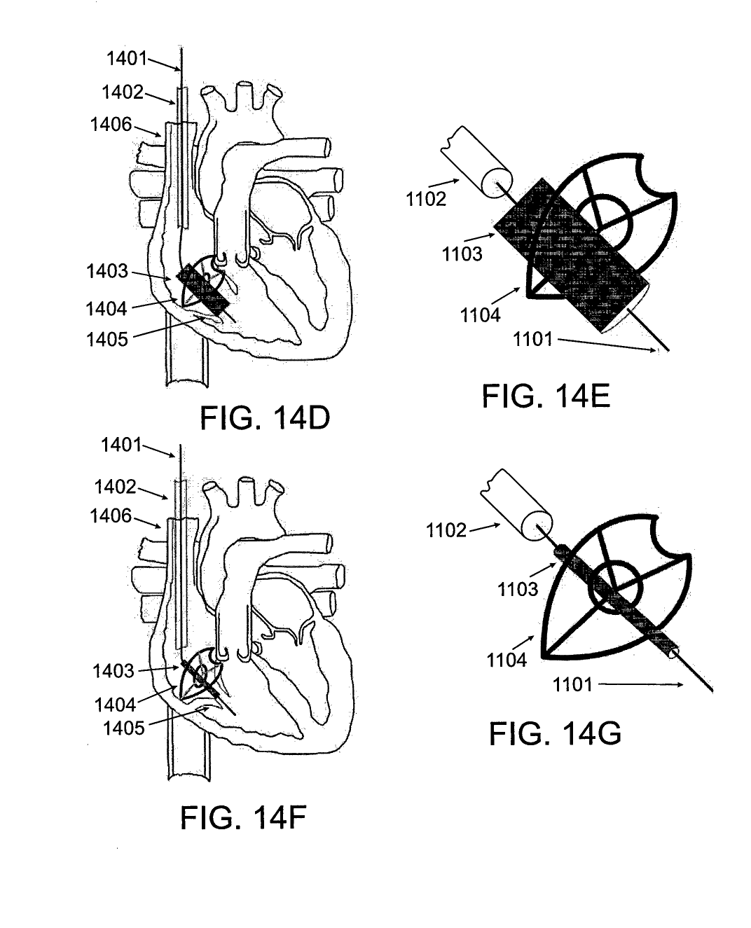

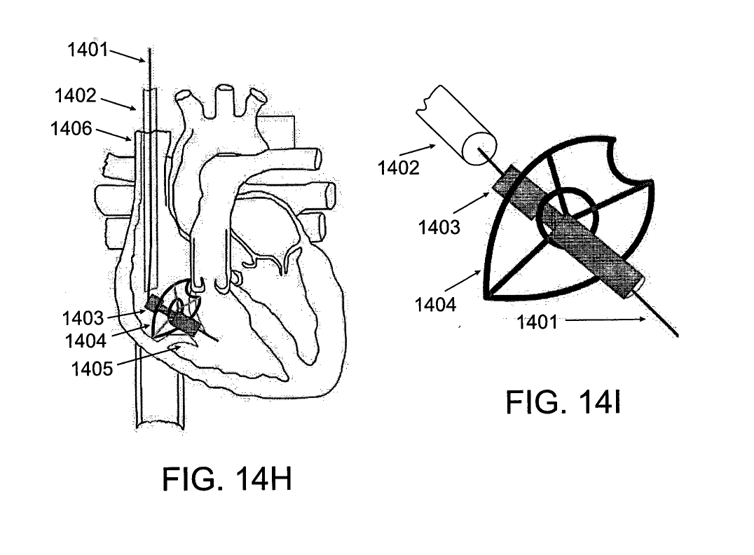

Once the apparatus is deployed in the tricuspid valve annulus, it may be adjusted, for example to better fit the annulus. An exemplary method for adjusting the implanted apparatus in a catheterization procedure using balloon inflation is described in the illustrations of FIG. 14.

In FIG. 14A 14B, 14D, 14F, 14H an anatomical structure of the heart is illustrated. FIG. 14C, 14E, 14G, 14I illustrate detailed enlargements of FIG. 14B, 14D, 14F, 14H respectively. Catheter 1402 is inserted into the superior vena cava 1406, in order to reach implanted apparatus 1404 (shown here in an embodiment that includes central adjustment sections, but may be of any other configuration as well). Catheter 1402 is inserted over guiding wire 1401 and balloon 1403 is inserted through the catheter, over the wire, to reach the apparatus implanted at the tricuspid valve annulus. A wire or multiple wires are guided through the different available orifices between the loops and rods of the apparatus 1404, for example six available orifices as shown in FIG. 14C, 14G, 14E. Balloon 1403 is inserted following the guiding wire and catheter to the chosen orifice, and then inflated. For example, in FIG. 14C a deflated balloon 1403 is inserted into an orifice of one of the main outer loops of the apparatus 1404. FIG. 14D and its enlargement 14E illustrate the balloon inflation within the chosen orifice, in this example causing expansion of the outer loop. FIG. 14F-14I depict the insertion followed by inflation of a balloon within an inner central adjustment section of the apparatus, possibly creating constriction of the respective main outer loop, if, for example, an common inner cord passes within both the inner adjustment section and main outer loop. Balloon inflation can be repeated several times for example in different available orifices of the apparatus to adjust it, for example to expand the main body of the apparatus by inflation within the main outer loops.

### Various Embodiments of Adjustment Units and Elements of an Annuloplasty Apparatus

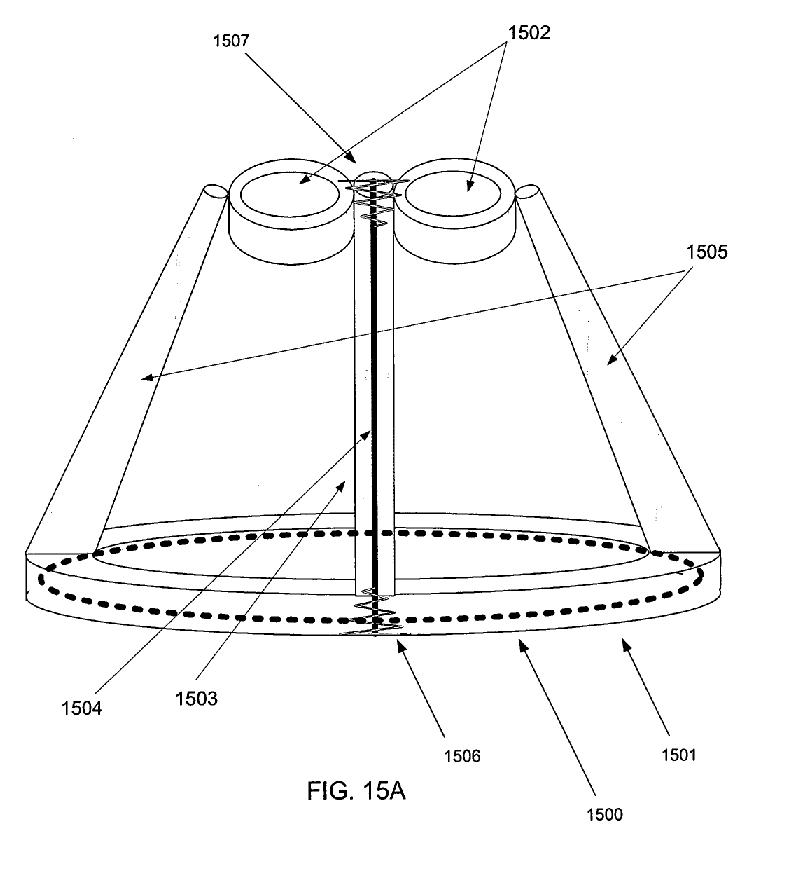

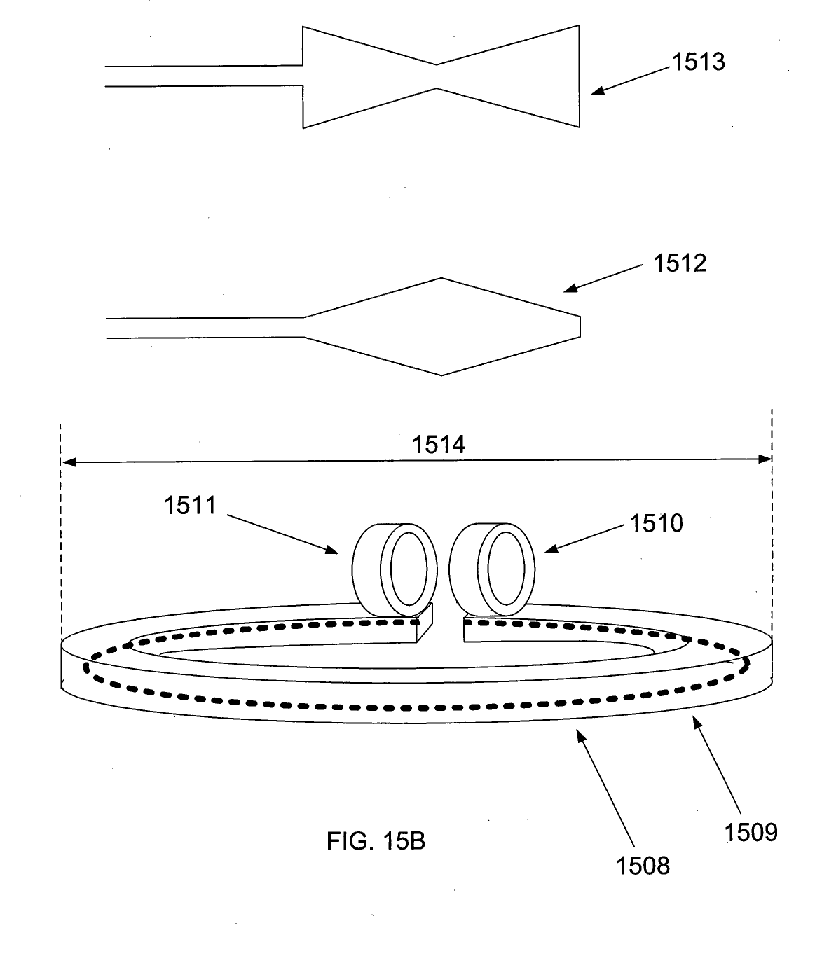

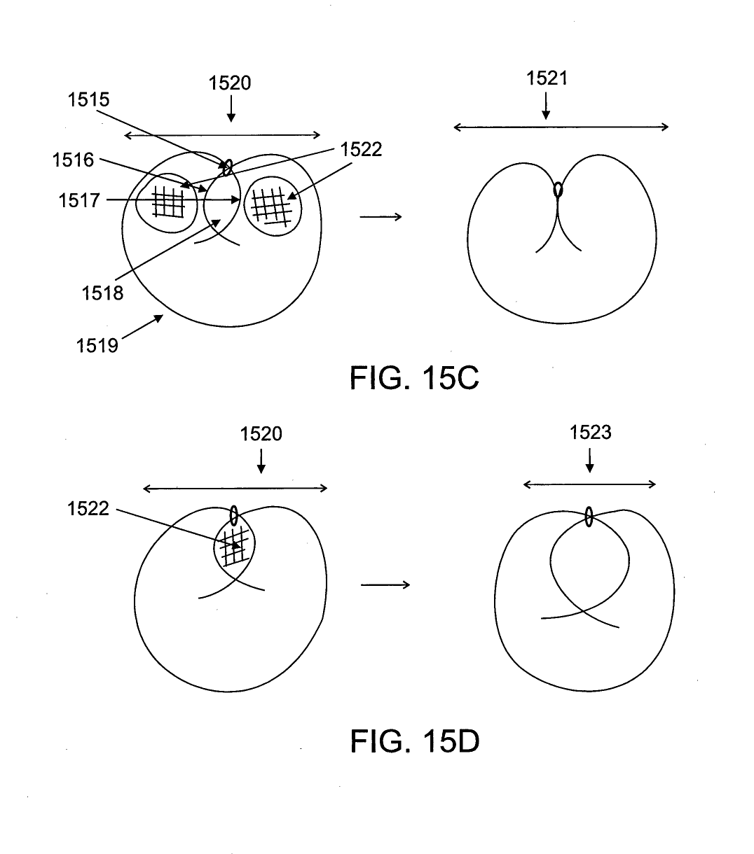

In some embodiments, designated adjustment sections are a component of, for example, a more complex adjustment unit. In some embodiments, the apparatus includes additional elements other than the designated adjustment section, that assist in adjusting the apparatus, for example constricting the main body. FIG. 15 shows some of these embodiments.

FIG. 15A shows a main body comprised of loop 1501 sized to fit a valve annulus, with an adjustment unit including adjustment loops 1502, an inner rod 1504, poles 1505 and lower and upper screws 1506, 1507. Two adjustment loops 1502 are mounted on poles 1505 which are connected to main loop 1501. Application of torque force to the adjustment loops 1502 is transferred by upper screw 1507, causing inner rod 1504, located inside the main rod 1503, to rotate. Each adjustment loop 1502 is capable of rotating inner rod 1504 in a certain direction (left rotation or right rotation). Rotation of inner rod 1504 causes spinning of lower screw 1506, possibly inducing expansion or constriction of the inner cord inside main loop 1501, and possibly expansion or constriction of the main loop. Optionally, one direction of rotation leads to expansion, and the other direction to constriction.

An adjustment unit that can be adjusted using inflation of special-shaped balloons is depicted is FIG. 15B. Annuloplasty apparatus 1508 includes a main body in the form of loop 1509, shown in this embodiment as a ring including a gap and having free ends. The apparatus includes two adjustment loops 1510 and 1511 positioned on a perpendicular plane to the main loop 1508. In order to adjust the size of main loop 1508, for example expand it, special-shaped balloons 1512, 1513 can be inflated within designated adjustment loop 1610 and 1511. For example, in order to reduce the diameter of main loop 1509, special shaped balloon 1513 is inserted into adjustment loops 1510, 1151. By inflating special shaped balloon 1513, the two inflated portions of it optionally push adjustment loops 1510, 1511 closer together, possibly reducing the diameter 1514 of main loop 1509. Similarly, in order to expand the diameter of main loop 1509, special shaped balloon 1512 is inserted into adjustment loops 1510, 1511. By inflating special shaped balloon 1512, adjustment loops 1510 and 1511 are pushed apart, possibly increasing diameter 1514 and therefore expanding main loop 1509. As shown in this embodiment, special shaped balloon 1513 has two triangular shaped ends, and special shaped balloon 1512 is formed in a diamond like shape.

As mentioned earlier, in some embodiments, special shaped balloons may be sized according to a designated adjustment section. Optionally, the shape of the special shaped balloons prevents the balloon from slipping out from an adjustment section.

An embodiment of the apparatus including an adjustment element in the form of a pivot pin 1515 is described in FIG. 15C and FIG. 15D. In FIG. 15C, pivot pin 1515 is located between the free ends of a loop segment, for example to bind the ends together to define an annular contour. Adjusting an apparatus that includes a pivot pin can be performed by applying force (such inflated walls of balloons 1522) on free ends 1516 and 1517.

Optionally, pivot pin 1515 serves as fastener or connector for binding the free ends of the loop segment and for maintaining a closed loop. Optionally, the pivot pin binds the loop ends in a movable manner, so as to allow the loops and/or inner cord to move or slide freely through the pivot. Optionally, the pivot pin is slideable along loops or loop ends as to allow its repositioning.

In order to expand main loop 1519 of the apparatus, balloons 1522 are inflated, pushing loop ends 1516, 1517 against each other, optionally decreasing overlapping area 1518 between the free ends. Optionally, the loop ends and/or an inner cord running through them slide within the pivot pin, possibly resulting in expansion of main loop 1519. Initial diameter 1520 is lengthened to a new diameter 1521.

In order to constrict main loop 1519, as seen in FIG. 15D, force in the opposite direction is used, applied from the within the section formed within the overlapping area of loop ends 1516 and 1517. Balloon 1522 can be inflated within the described section, thereby increasing the overlapping area between loop ends 1516, 1517. Optionally, the loop ends and/or an inner cord running through them slide within the pivot pin, possibly resulting in constriction of main loop 1519. Initial diameter 1520 of main loop 1519 is shortened to a smaller diameter 1523.

### An Embodiment of an Annuloplasty Apparatus Including a Telescopic Pole

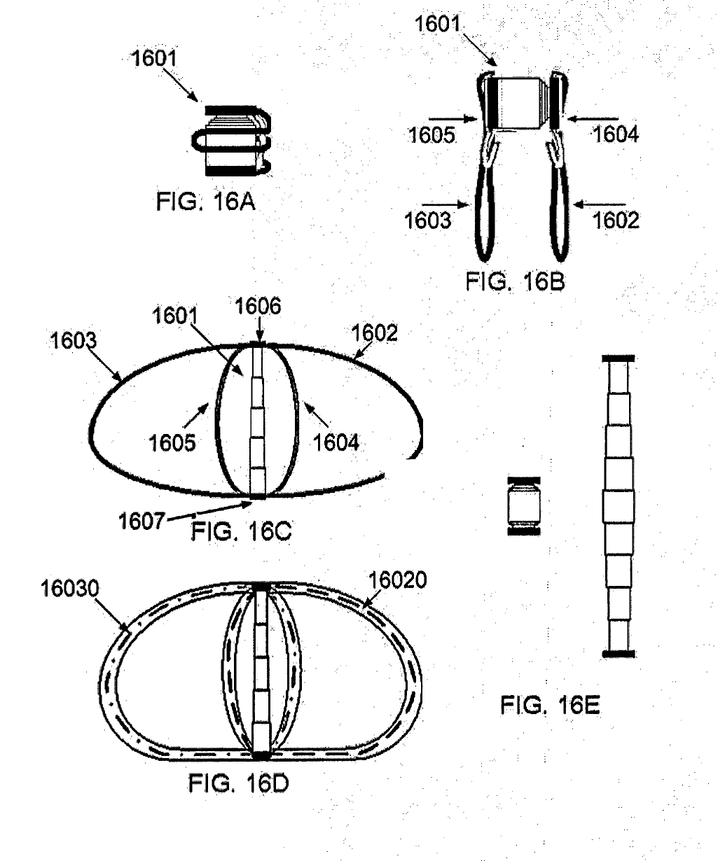

A rigid member of the annuloplasty apparatus, in some embodiments, includes at least a segment of a telescopic pole. FIG. 16 shows an apparatus comprising a rigid member which is a telescopic pole, in accordance with an exemplary embodiment of the invention. Optionally, telescopic pole 1601 is shortened, as shown in FIGS. 16A and 16B, for example during insertion to the heart, to create a compact form which can be delivered through a catheter. Optionally, telescopic pole 1601 is lengthened, for example during deployment at the valve annulus. Optionally, telescopic pole 1601 is lengthened in the direction of one of its ends (as seen in FIG. 16A-16D) or in the direction of both ends, as seen in FIG. 16E.

In some embodiments, during insertion to the heart, loops 1602-1605 comprising the main body and adjustment sections of the apparatus are folded over shortened telescopic pole 1601 to create a compact structure, as shown in 16A, or folded along the sides of the pole to create a butterfly-like structure, as shown in 16B.

In some embodiments, ends 1606, 1607 of telescopic pole 1601 also serve as connectors for attaching the loops or loop segments to telescopic pole 1601 in a non-rigid, movable manner, to allow loops and inner cord (16020, 16030, shown in FIG. 16D) to slide through the connector when adjustments are taking place. Optionally, when no force is applied, the connectors assist in maintaining the current length of telescopic pole 1601.

A general view of the apparatus with a fully lengthened telescopic pole is shown in 16C. After implantation, the length of telescopic pole 1601 may be adjusted in a similar method to adjusting the loops of the apparatus. For example, applying force (for instance by using the walls of an inflated balloon) inside adjustment section 1605 will possibly lengthen telescopic pole 1601. Optionally, the length of telescopic pole 1601 is adjusted by pushing or pulling on the connectors on both ends of the pole, for example using a catheter.

In some embodiments, the apparatus comprises an adjustment section that is coupled to the pole, for example formed as a cable extending between the ends of the pole. Optionally, a balloon is inflated in the section to extend the pole. In some embodiments, the apparatus comprises a plurality of telescopic poles, one or more of which are coupled to adjustment sections. In an example, 4 telescopic poles extend for example from a center of the main ring, forming a cross configuration. Optionally, the poles are extendible to varying lengths.

### Various Embodiments of an Annuloplasty Apparatus Including a Restraining Member

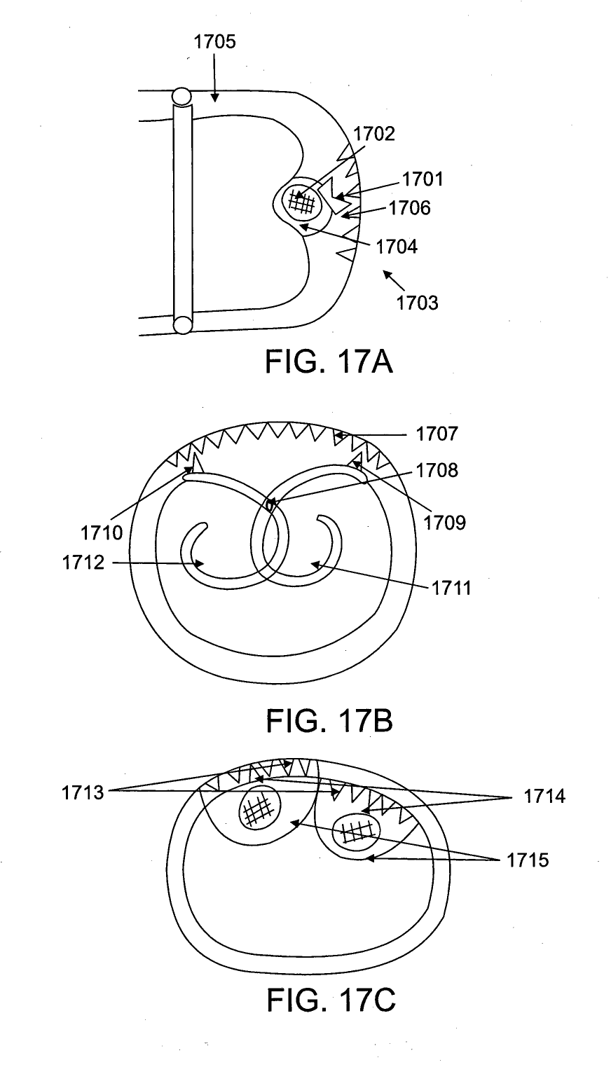

FIG. 17 shows various embodiments of the apparatus comprising restraining members that can be incorporated to different parts of the apparatus, such as the main body or adjustment section, in accordance with an exemplary embodiment of the invention. Optionally, a restraining member maintains a current configuration of the apparatus, for example of the main body. Optionally, a restraining member is used to assure that a newly obtained shape or size will maintain for an unlimited period of time or until adjustments are made. Optionally, a restraining member allows an adjustment of only one direction or type (for example expansion or constriction), or limits an adjustment to a predefined extent, for example to a predefined diameter. Optionally, previously described elements such as the structural crossing element, the connector or the inner cord function as restraining members, for example a connector which clasps on the inner cord to prevent its movement. Optionally, the restraining member comprises a lock piece which is released (or configured to an open position), for example prior to adjustments.

FIG. 17A depicts a restraining member including a lock piece in the form of a pawl 1701. Optionally, pawl 1701 intermeshes a toothed rack 1706, mounted for example in an outer loop or in an adjustment section, for example mounted to the inner cord. Optionally, pawl 1701 is shaped to engage the rack in way that normally blocks its movement on the rack, until moved from within the toothed rack or pushed to a position that allows it to advance on the rack in a single direction (ratchet-like mechanism).

In some embodiments, pawl 1701 is used to maintain the existing shape or size of the apparatus, for example maintaining the size of the main body. Applying force on pawl 1701, such as by inflating a balloon within designated adjustment section 1704, may push pawl 1701 away from the toothed rack 1706, or alternatively switch its position to allow its advancement on the rack.

When pawl 1701 no longer limits movement, it is possible to adjust, for example, the main outer loop 1705 of the apparatus, for example to expand it.

Optionally, the configuration of pawl 1701 changes as a result of applying force. Optionally, the pawl is moved from the toothed rack or alternatively pushed to a movable position only by applying a certain direction and/or magnitude of force, for example to avoid unintentional adjustments. Optionally, balloon 1702 is re-inflated after the required adjustment is performed (for example expansion of main outer loop 1705) in order to push pawl 1701 back onto the toothed rack to its original position to limit movement.

An embodiment involving the use of a toothed rack 1707, pawls 1709 and 1710, and a pivot point 1708 as previously described is presented in FIG. 17B. Toothed rack 1707 is mounted in the main outer loop or in a segment of it. Pawl 1709 engages toothed rack 1707 in a way that allows pawl 1709 to advance on the rack in a single direction only, for example enabling expansion of the loop only. Similarly, pawl 1710 positioned opposite to pawl 1709 may enable constriction adjustments only. Optionally, the pawls are positioned within separate adjustment sections 1711 and 1712. Optionally, the apparatus described in this figure is adjusted in a like manner to the adjustment described above in FIGS. 15C and 15D, but possibly involves the pawls advancing along the toothed rack, for example using the method described in FIG. 17C below.

FIG. 17C depicts an embodiment comprising a set of two toothed racks with pawls as previously described, where the pawls can be actively forced to advance along the rack. Optionally, force is applied, for example by inflating and deflating a balloon multiple times, in order to incrementally advance the pawl on the rack. As shown in this embodiment, two opposite direction pawls 1714 and two parallel toothed racks 1713 are used, for example one for advancing in every direction, to allow expansion in one direction and constriction in the opposite direction. Optionally, two separate adjustment sections 1715 are used, one for each type of adjustment. Optionally, each section allows accessing a certain toothed rack only. Optionally, one section is used for releasing the lock, for example by balloon inflation, and the other section is used, for example, for expanding the main body.

### An Embodiment of an Annuloplasty Apparatus Including Peripheral Hooks for Anchoring the Main Body of the Apparatus to Body Tissue at the Valve Annulus

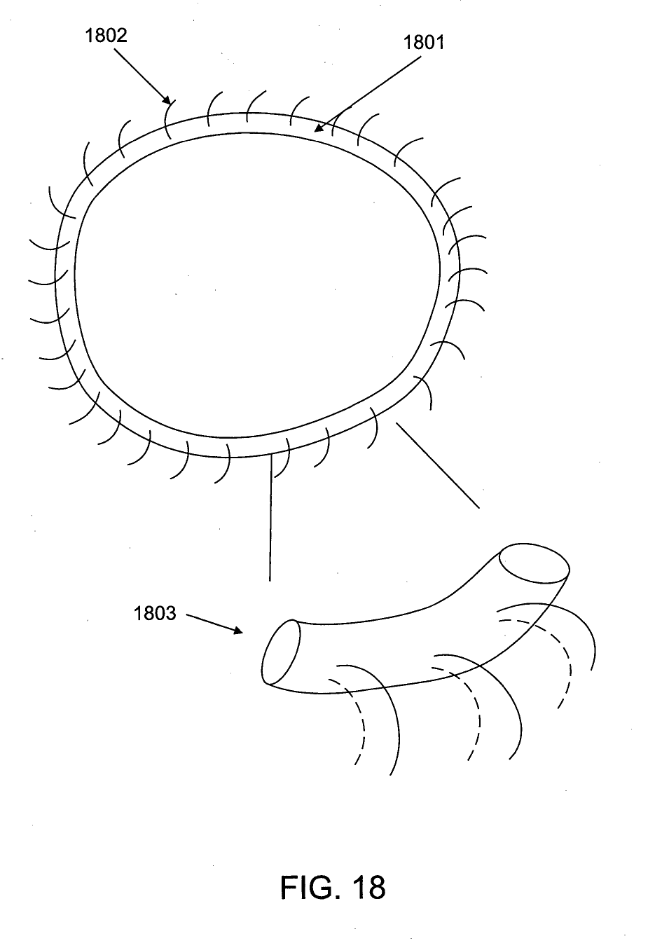

FIG. 18 shows a general view of an embodiment of an annuloplasty apparatus, where the main body in the form of loop 1801 includes peripheral hooks 1802 for attaching the apparatus to body tissue and fixing it in the required position in vivo, according to some embodiments of the invention. Optionally, during manipulation of the apparatus to reach the valve annulus, hooks 1802 remain in a folded or collapsed position, attached to main loop 1801. When the apparatus is shaped to its deployed configuration, the shaping taking place for example within the left atrial cavity, the hooks assume an open configuration. Optionally, when main loop 1801 is expanded, the hooks bend towards the tissue and clasp in it, as seen in the dotted lines of the enlarged view 1803 of a segment of the loop. The radial force applied on loop 1801 in order to expand it (such as the force applied by the walls of an inflated balloon) can be utilized for pushing hooks 1802 in the direction of the tissue, anchoring the apparatus to the body tissue at the intended location.

In order to reshape the apparatus after the initial deployment, for example to constrict it, a mechanism for uncoupling the hooks from the loop can be used. In an exemplary mechanism, the catheter (or insertion arm) guiding the apparatus into position is used for uncoupling and/or re-coupling the hooks to the loop. In another exemplary mechanism, applying radial force stronger than a pre-defined threshold, for example by inflating a balloon to a certain pressure, will cause the hooks to bend and clasp the tissue, while applying weaker radial force will allow adjusting the apparatus.

Optionally, at the end of the adjustment, sutures are used to fasten the hooks into place. In some embodiments, the hooks are made of a long lasting biocompatible material, for example titanium.

### Additional Embodiments of an Adjustable Ring Type Apparatus and Methods of Adjustment

According to some embodiments, an adjustable constraining apparatus comprising a main body and a designated adjustment section is implanted in the intratrial septum of the heart, for example to control blood flow shunting across from the right atrium to the left atrium, for example in the case of severe pulmonary hypertension.

Optionally, the apparatus, for example its main body, is sized to maintain an opening between the right and left atrium, for example by sizing the apparatus to a diameter between 1 to 10 mm. Optionally, the adjustment section is positioned externally to the main body, for example externally to the opening, to allow partial or full closure of the opening, for example by constricting a main loop of the apparatus. The adjustment section is positioned, for example, inside the right atrium. Optionally, blood flow shunted from the right atrium to the left atrium is controlled by adjusting a diameter of the main body of the apparatus, possibly affecting cardiac output and hypoxemia. In an exemplary embodiment of the invention, this allows gradual (e.g. stepwise) closure of the opening, thereby preventing shock to the heart during sudden change in flow dynamics therethrough.

In some embodiments, mechanisms such as the implantation mechanism and/or adjustment mechanism that were previously described, are utilized. Optionally, the apparatus is implanted in the intra-atrial septum in a minimally invasive procedure, such as a transcatheter procedure, optionally through the transapical route. Optionally, the implanted apparatus is adjusted by applying mechanical force when contacting an adjustment section of the apparatus. Optionally, a balloon is inflated in the designated adjustment section, the walls of the inflated balloon applying force on the adjustment section or a part of it. Optionally, the apparatus is adjusted in a minimally invasive procedure, such as catheterization.

According to some embodiments, an constraining apparatus with a design utilizing components as described above is implanted in organs other than the heart, such as the digestive system. Optionally, the apparatus is implanted in the digestive system in order to reduce the size of a passage. Optionally, the apparatus is implanted during bariatric surgery, for example to resize the passage of a gastric inlet leading to the stomach. Optionally, the main body of the apparatus is sized according a gastric inlet, for example having a diameter of 3 cm. Optionally, the adjustment section is positioned externally to the main body, for example to keep a clear passage through the main body.

Optionally, the apparatus is implanted in the gastric inlet in a minimally invasive procedure, such as a laparoscopic procedure. Optionally, the implanted apparatus is adjusted by applying mechanical force when contacting an adjustment section of the apparatus. Optionally, a balloon is inflated within the designated adjustment section, the walls of the inflated balloon applying force on the adjustment section or a part of it. Optionally, the apparatus is adjusted in a minimally invasive procedure, such as laparoscopy. Optionally, the apparatus is adjusted according to the changing needs of a patient, for example to enlarge or to reduce the size of a passage to the stomach. Optionally, two designated adjustment sections (e.g. extending away from or parallel to the stomach) are provided, one to expand the ring and one to constrict the ring.

According to some embodiments, an annuloplasty apparatus implanted at the heart valve annulus (such as the mitral valve annulus and/or the tricuspid valve annulus) as previously described in this application is optionally used as a platform for an artificial heart valve, for example a mechanical heart valve. According to some embodiments, the annuloplasty apparatus is implanted in order to repair the structure and functioning of the valve annulus, and at a later stage (for example 2 years post implantation), according to the patient's needs, the natural valve may be replaced by a prosthetic valve. Optionally, replacing the natural valve includes implanting artificial leaflets, such as 1, 2 or 3 leaflets. Optionally, components of the artificial valve such as semicircular leaflets are constructed within the lumen of the existing main body of the apparatus. Optionally, the leaflets are attached to the main body using struts or any other binding element. Optionally, the struts allow rotation of the leaflets for controlling blood flow through the valve. Optionally, the prosthetic leaflets are made of a biocompatible material, for example stainless steel or titanium.

In some embodiments, the annuloplasty apparatus is adjustable, for example constrictable, after the addition of artificial valve components such as the leaflets. Optionally, the proposed method of adjustment, for example using a balloon catheter, is used for deploying the leaflets in the optimal configuration as well.

In some embodiments, a cardiac valve uses the mechanism as described above for controlling the shape and/or size of an annulus thereof, for example, the annulus of the artificial valve being in the form described above for an annuloplasty device.

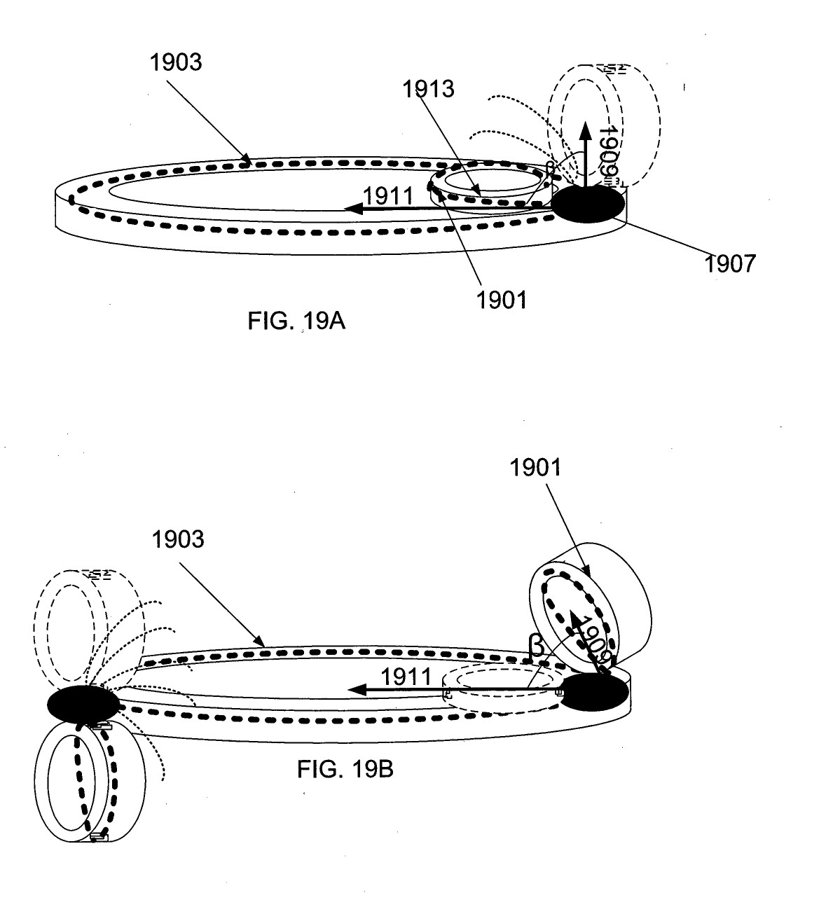

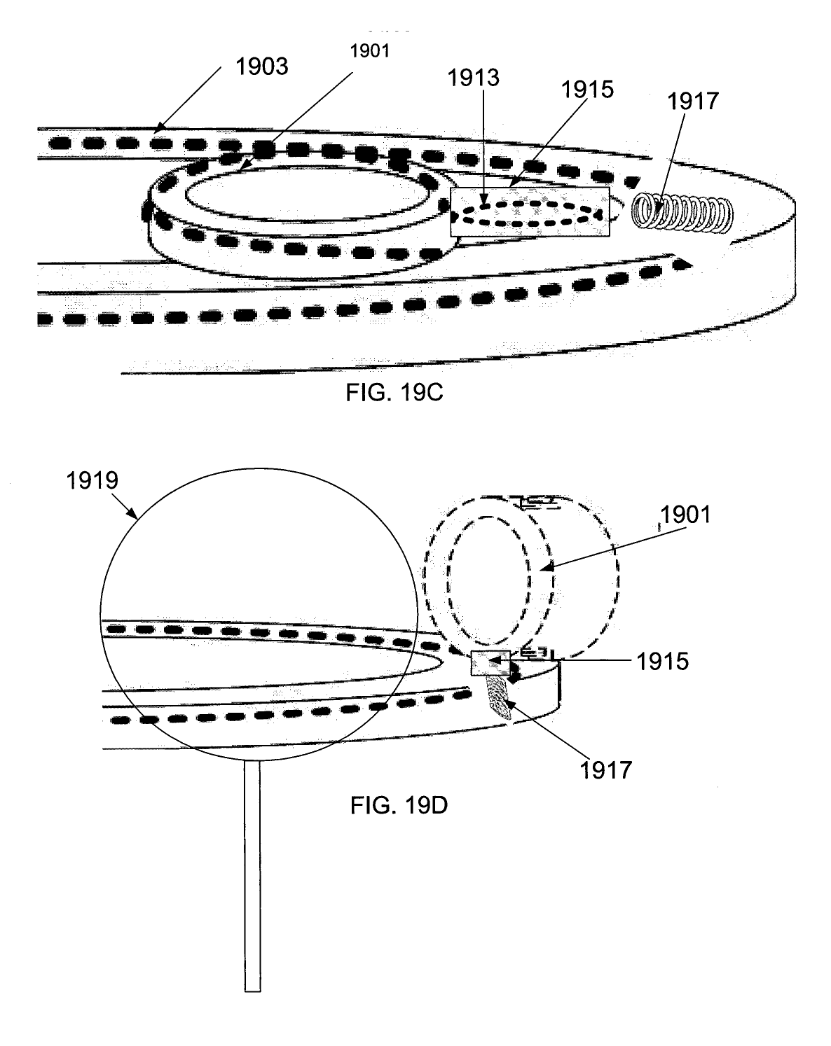

FIGS. 19A-B show an annuloplasty apparatus comprising one or more adjustment sections that are positionable and/or re-positionable with respect to the valve annulus and/or with respect to a main ring of the apparatus, according to some embodiments of the invention.

FIG. 19A shows an apparatus comprising a single adjustment section, FIG. 19B shows an apparatus comprising two adjustment sections, for example positioned opposite each other. In various embodiments the apparatus may include a plurality of adjustment sections, such 2, 3, 4, 5, 6 or a higher number of adjustment sections.

In some embodiments, for example as shown in FIG. 19A, an adjustment section for example formed as loop 1901 is movable, for example with respect to main ring 1903. Optionally, adjustment section 1901 is rotated around an axis, for example being a connection point 1907 between the main ring 1903 and adjustment section 1901. Optionally, connection 1907 allows moving the adjustment section 1901, for example by including a hinge. Optionally, the hinge comprises a “click-type” mechanism for step-advancing section 1901 to a certain position.

In some embodiments, adjustment section 1901 is movable from a position in which it lies within a plane defined by at least a portion of main ring 1903, to a position in which it is substantially perpendicular to that plane. A plane defined by at least a portion of the main ring may include, for example, a plane defined by an arc segment of main body 1903, for example an arc with a central angle ranging between 5-120 degrees, 20-200 degrees, 60-300 degrees. Optionally, adjustment section 1901 is movable in a range between an upright position, for example extending in a proximal direction, such as towards the left atrium, to a position in which the section levels with a plane defined by the main ring, and/or to a position in which the adjustment section extends distally, such as towards the left ventricle, for example as shown in FIG. 19B.

In some embodiments, a position of adjustment section 1901 with respect to main ring can be described using a set of vectors 1909 and 1911. Vector 1909 extends between connection point 1907 and a center of adjustment section 1901 (for clarity purposes, the vector is shown with respect to the moved position of the section, indicated by the dashed lines). Vector 1911 extends between connection point 1907 and a center of main ring 1903. Optionally, adjustment section 1901 can be positioned at a certain angle 13, formed between the two vectors. The selected angle 13 may range, for example, between 0-120 degrees, such as 30 degrees, 60 degrees, 85 degrees, or intermediate, larger or smaller angles.

FIG. 19B shows, for example, adjustment section 1901 being positioned in an intermediate position, at angle β of, for example, 70 degrees between vectors 1909 and 1911. Similarly, an angle β may be formed with respect to the opposite, ventricular facing side of main body 1903.

In some embodiments, adjustment section 1901 is formed of a flexible material or a semi-flexible material, such as polyacetal, polyethylene, acetal resin or other elastic polymer, enabling flexing and/or deforming of the loop with respect to main ring 1902.

In some embodiments, connection point or segment 1907 comprises a restricting element, such as a clasp or pin, adapted to limit movement of the adjustment section. For example, the restricting element may enable movement of adjustment section 1901 in only one direction, such as towards the atrium, and prevent movement of section 1901 in a second direction, such as towards the ventricle.

In some embodiments, adjustment section 1901 is selectively positioned, for example by a physician. Optionally, force is applied to move the adjustment section, for example using a catheter and/or a balloon. Additionally or alternatively, force is applied by an element that is positioned within main ring 1903, for example a balloon implantable valve. Additionally or alternatively, adjustment section 1901 moves in response to an adjustment of a different portion of the apparatus, such as the main body.

Optionally, adjustment section 1901 is elastically deformed to a position with respect to main ring 1903. Optionally, the section returns back to its original position when force is no longer applied. Alternatively, the section is maintained in the new position, for example by a connector suitable for fixating the section with respect to the ring, for example at connection point 1907.

In some embodiments, adjustment section 1901 is selectively positioned with respect to main ring 1903, for example to enable inflation of a balloon within the main ring. Optionally, section 1901 extends away from the main ring 1903, such as towards the volume of the atrium or towards the volume of the ventricle, so that it does not interfere with inflation.

In some embodiments, adjustment section 1901 is positioned parallel to the direction of blood flow, for example of blood passing through the annulus, to reduce interference with the flow. In some embodiments, the adjustment section is dynamically positioned, for example its position can be set by the flow. Additionally or alternatively, the adjustment section is positioned and/or sized and/or shaped to divert or otherwise affect flow of blood, for example positioned to encourage flow through the valve.

In some embodiments, adjustment section 1901 is shaped and/or sized to reduce or prevent damage to tissue, such as wall tissue of the atrium. Optionally, adjustment section 1901 is small enough so that it does not interrupt the functioning of the valve's leaflets. For example, a diameter of adjustment section 1901 ranges between 0.1 cm-2 cm.

In some embodiments, adjustment section 1901 comprises an inner cable 1913. Optionally, inner cable 1913 is couples between adjustment section 1901 and main ring 1903. Optionally, a segment of inner cable 1913 running through adjustment section 1901 is long enough to enable expanding main ring 1903. A potential advantage of the ability to tilt adjustment section 1901 away from main ring 1903 may include using a larger adjustment section 1901, for example with respect to an apparatus in which the adjustment section lies within the lumen of the main ring and would need to be limited in size to reduce interfering with valve functioning.

In some embodiments, adjustment section 1901 is covered by a material suitable for softening contact between section 1901 and wall tissue, for example reducing the risk of mechanical damage such as scratching of the tissue. Optionally, the material is elastic enough to enable performing one or more adjustments of the apparatus, without breaking or tearing. Optionally, the material is selected to avoid growth of biological material on the apparatus, thereby reducing the risk of thrombus. Exemplary material may include a soft plastic, such as Dacron.

FIGS. 19C-D show an exemplary movement mechanism of the adjustment section, according to some embodiments of the invention. In some embodiments, adjustment section 1901 is attached to the main ring by a tab 1915. Optionally, tab 1915 is formed with one or more channels to accommodate inner cable 1913.

In some embodiments, main ring 1903 is formed with a slot for receiving tab 1915. Optionally, tab 1915 is coupled, on one end, to adjustment section 1901, and on the other end to an elastic member such as a spring 1917.

In some embodiments, for example when a balloon 1919 is inflated within ring 1903, tab 1915 is pushed into its slot in main ring 1903, compressing spring 1917. Optionally, when tab 1915 is pushed in, adjustment section 1901 is bounced to extend away from main ring 1903, for example in the atrial direction.

Optionally, when balloon 1919 is collapsed, and compression force on spring 1917 ceases, the spring extends back to an initial length, pushing tab 1915 back out into the lumen of main ring 1903, thereby causing adjustment section 1901 to move back to an initial position.

Other embodiments may include various mechanisms for moving the adjustment section with respect to the main ring, for example including coupling the adjustment section to a cylinder that is threaded onto the main ring and serves as a hinge, using a fastener that enables rotation, and/or other mechanisms which couple the adjustment section to the ring in a movable manner.

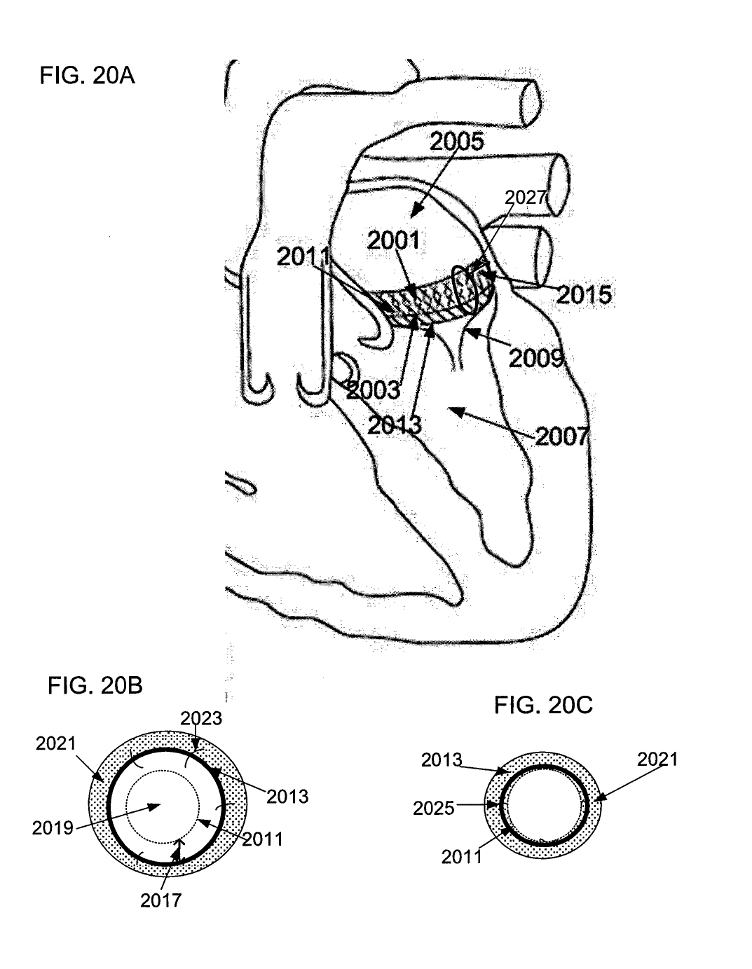

FIG. 20A illustrates a prosthetic mitral valve 2001 and an annuloplasty apparatus 2003 implanted together in a heart, according to some embodiments of the invention.

In some embodiments, prosthetic valve 2001 is introduced into the heart, and positioned over or in place of the natural mitral valve, between the left atrium 2005 and left ventricle 2007.

In some embodiments, prosthetic valve 2001 is a biological valve, an artificial valve, or any other type of valve.

In some embodiments, prosthetic valve 2001 comprises a set of leaflets 2009. Optionally, prosthetic valve 2001 comprises a mesh stent-like body 2011, which supports leaflets 2009 and may assist in holding the leaflets in place. (The mesh stent like body may resemble a mesh stent body commonly used in prosthetic aortic valves). Optionally, the supporting body 2011 of the prosthetic valve 2001 is cylindrical.

In some embodiments, annuloplasty apparatus 2003 is implanted prior to prosthetic valve 2001. Alternatively, annuloplasty apparatus 2003 is implanted following valve 2001. Optionally, annuloplasty apparatus 2003 is positioned externally to valve 2001, for example circumferentially surrounding the valve.

Optionally, leaflets 2009 of valve 2001 are attached within the lumen of main ring 2013 of the annuloplasty apparatus. Optionally, adjustment section 2015 of the annuloplasty apparatus is moved away from the plane defined by main ring 2013, for example towards atrium 2005, so as the reduce interference with leaflets 2009.

In some embodiments, annuloplasty apparatus 2003 holds valve 2001 in place. Optionally, by constricting main ring 2013 around supporting body 2011, movement of valve 2001, such as movement in the atrial direction or movement in the ventricular direction is reduced or prevented by ring 2013. In some embodiments, main ring 2013 is attached to tissue of the walls of the annulus, for example attached at one or more circumferential locations, for example by sutures and/or hooks. Optionally, by closely surrounding supporting body 2011 of the valve, main ring 2013 anchors valve 2001 in place. A potential advantage of positioning an annuloplasty apparatus to encircle a prosthetic valve may include the ability to use a valve having a supporting body, such as a stent-like cylindrical body, in the mitral valve. It is noted that prosthetic valves having a stent like supporting body are sometimes used as a tricuspid valve, but are less commonly used as a mitral valve due to a lack of anchoring locations to the tissue. Optionally, an annuloplasty apparatus positioned to surround the supporting body of the valve provides sufficient anchoring to reduce or prevent the valve from moving.

In some embodiments, for example during implantation, supporting body 2011 of valve 2001 is positioned with main ring 2013. Optionally, supporting body 2011 is expanded, for example by inflating a balloon within a lumen of supporting body 2011. Optionally, by inflation of body 2011, radially outward force is applied to ring 2013, optionally causing the ring to expand as well. In some embodiments, for example after the balloon is collapsed and/or removed, ring 2013 is constricted, for example by expanding adjustment section 2015, to fit body 2011.

In some embodiments, supporting body 2011 is rigid enough to withstand the constricting force applied by ring 2013, for example during constriction of main ring 2013, to maintain an opening, for example an opening having a diameter large enough to allow sufficient blood flow through. Optionally, supporting body 2011 limits the constriction of ring 2013 so that movement of leaflets 2009 is not interrupted.

In some embodiments, ring 2013 and supporting body 2011 are coupled to each other by sutures, hooks and/or other fixation devices.

In some embodiments, adjustment section 2015 extends a radial distance 2027 away from an inner wall of supporting body 2011, for example extending a distance ranging between 0.1 mm-3 cm. Optionally, adjustment section 2015 extends in the atrial direction beyond supporting body 2011.

FIGS. 20B and 20C are cross sections of main ring 2013 of the annuloplasty apparatus, and supporting body 2011 of the prosthetic valve. In some embodiments, main ring 2013 is adjusted to fit at least a portion of valve 2001, such as supporting body 2011. Optionally, for example as shown in FIG. 20C, main ring 2013 is constricted to match a contour of body 2011. Optionally, a tight fit is obtained, reducing the risk of paravalvular leaks which may occur within one or more gaps 2017, for example as shown in FIG. 20B, between supporting body 2011 and main ring 2013 before the adjustment. Additionally or alternatively, supporting body 2011 is expanded or constricted to fit main ring 2013 of the annuloplasty apparatus. Optionally, annuloplasty apparatus 2003 and/or prosthetic valve 2001 are adjusted to match an anatomy of the valve annulus 2019.

In some embodiments, main ring 2013 is attached to tissue 2021, for example by one or more sutures and/or hooks 2023. Optionally, when main 2013 is constricted around supporting body 2011, tissue 2021 is slightly stretched in a radially inwards direction. Additionally or alternatively, supporting body 2011 is expanded until contacting ring 2013. Optionally, when a desired positioning of valve 2001 and/or of annuloplasty apparatus 2003 with respect to each other and/or with respect to the tissue is obtained, supporting body 2011 and main ring 2013 are coupled to each other, for example by one or more sutures or clasps 2025.

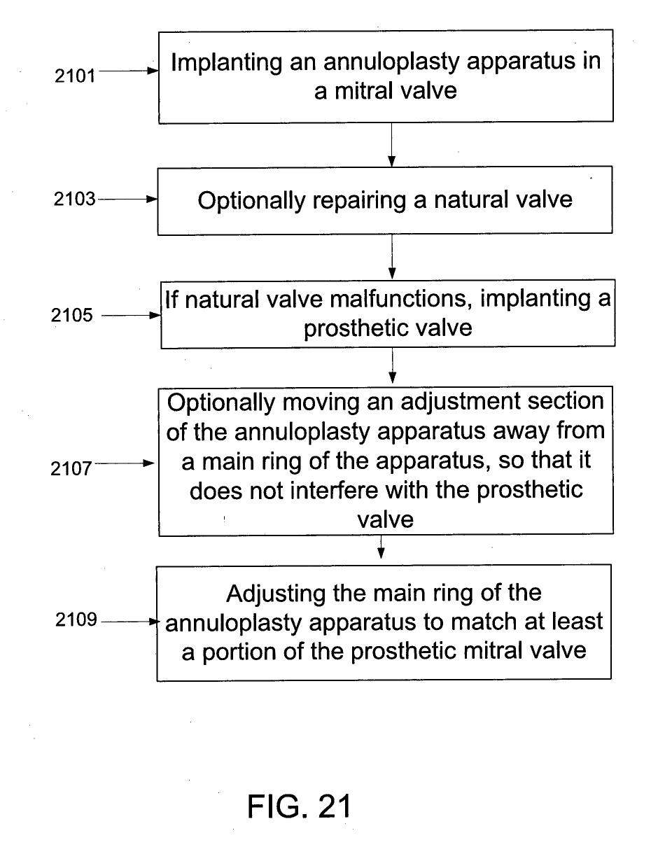

FIG. 21 is a flowchart of a method of using an annuloplasty apparatus together with a prosthetic mitral valve, according to some embodiments of the invention.

In some embodiments, an annuloplasty apparatus is implanted (2101) in a mitral valve. Optionally, an attempt is made, for example using surgical techniques and the annuloplasty apparatus, to preserve the natural valve (2103).

In some embodiments, if natural valve functioning is not repaired, a prosthetic valve is implanted (2105). Optionally, the prosthetic valve is implanted a certain time period after the implantation of the annuloplasty apparatus, for example, days, weeks, months, or even years after the annuloplasty apparatus was implanted. In some embodiments, the prosthetic valve is implanted percutaneously, for example through a transapical approach.

In some embodiments, the prosthetic valve comprises leaflets and optionally a supporting stent-like body.

Optionally, an adjustment section of the annuloplasty apparatus extends away from the main ring, for example moved in the direction of the atrium (2107), so as to reduce interference with the prosthetic valve, for example with the valve's leaflets, which are positioned within the annulus.

In some embodiments, the annuloplasty apparatus is then adjusted (2109), for example by expanding or constricting a main ring of the apparatus, to match at least a portion of the valve, for example to tightly fit around the supporting body of the valve. Additionally or alternatively, the supporting body of the valve is expanded or constricted to match the contour of the main ring.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is expected that during the life of a patent maturing from this application many relevant annuloplasty apparatuses will be developed and the scope of the term annuloplasty apparatus is intended to include all such new technologies a priori.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures, for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1. An adjustable implantable annuloplasty apparatus comprising:
a ring-shaped main body aligning at least 50% of a human heart valve annulus; a designated adjustment section for adjusting said main body;
wherein said main body is shaped so that a lumen of said main body of a deployed apparatus coincides with a lumen of said heart valve annulus, and wherein at least a part of said adjustment section extends from said main body; wherein said main body and said designated adjustment section of a deployed apparatus are fully contained within a 5 cm diameter; and
wherein a position of said designated adjustment section with respect to a plane defined by at least a portion of said ring-shaped main body is controllable.

2. The apparatus according to claim 1, wherein said main body and said designated adjustment section are mechanically adjustable in vivo.

3. The apparatus according to claim 1, wherein said designated adjustment section extends within said lumen of said main body.

4. The apparatus according to claim 3, wherein said designated adjustment section occupies only a portion of said lumen of said main body during adjustments to maintain an opening in which blood can flow through.

5. (canceled)

6. The apparatus according to claim 1, wherein said designated adjustment section is in the form of a loop.

7. The apparatus according to claim 6, wherein said loop is structured as a hollow tube.

8. The apparatus according to claim 7, wherein said loop comprises an inner cord passing within said hollow tube of said loop.

9-11. (canceled)

12. The apparatus according to claim 1, wherein said apparatus is collapsible to be delivered to the heart in a transapical catheterization procedure.

13. The apparatus according to claim 1, wherein said adjustment section includes at least one radiopaque marking for visualizing said adjustment section using a fluoroscope.

14. (canceled)

15. The apparatus according to claim 1, wherein a lumen is formed between said designated adjustment section and said main body, said lumen sized to receive a balloon.

16. The apparatus according to claim 1, wherein said main body comprises a plurality of hooks for anchoring said main body to the tissue of said human heart valve annulus.

17. The apparatus according to claim 1, wherein at least one of said main body and said adjustment section comprises a restraining member for maintaining the shape of said main body.

18-21. (canceled)

22. The apparatus according to claim 21, wherein said main body comprises a ventricular facing side and an opposing atrial facing side, and said designated adjustment section extends away from said main body towards the volume of the atrium.

23. The apparatus according to claim 22, wherein said adjustment section is formed, at least in part, of a soft flexible material which reduces damage to tissue the section may come in contact with.

24-36. (canceled)

37. A method for mechanically adjusting an implanted adjustable annuloplasty apparatus post implantation, comprising:
engaging an adjustment section of said apparatus inside the heart using a tool suitable for applying mechanical force; and
adjusting a main body of the apparatus by applying mechanical force, thereby reshaping the heart valve annulus post implantation.

38-42. (canceled)

43. An adjustable implantable annuloplasty apparatus comprising:
a ring-shaped main body aligning at least 50% of a human heart valve annulus;
a designated adjustment section for adjusting said ring-shaped main body; said designated adjustment section is controllable;
wherein said ring-shaped main body is shaped so that a lumen of said ring-shaped main body of a deployed apparatus coincides with a lumen of said heart valve annulus, and wherein at least a part of said adjustment section extends from said ring-shaped main body; wherein said main body and said designated adjustment section of a deployed apparatus are fully contained within a 5 cm diameter;
wherein the apparatus is adjustable multiple times post implantation.

44. The apparatus of claim 43, wherein the designated adjustment section is controllable to constrict and expand.

45. The apparatus of claim 43, wherein said apparatus is collapsible to be delivered to the heart in a transapical catheterization procedure.

46. The method of claim 37, further comprising:
adjusting a ring-shaped main body using a designated adjustment section to match a contour of a mitral valve.

47. The method of claim 46, wherein said adjusting comprises expanding or constricting said main loop of said annuloplasty apparatus.

48. The method of claim 37, wherein said adjusting the main body of the apparatus is performed at least 1 month post surgery.
